(19) United States
(12) Patent Application Publication
(10) Pub. No.: US 2025/0285178 A1
(43) Pub. Date: Sep. 11, 2025

(54) ULTRAVIOLET CLEANING OF CARRIERS IN A DRIVE-UP BANKING SYSTEM

(71) Applicant: Hamilton Safe, Mason, OH (US)

(72) Inventors: V. John Stroia, Loveland, OH (US); Todd Lefevers, Hamilton, OH (US); William Harvey Detherage, Amelia, OH (US); Brian Miller, Milford, OH (US)

(21) Appl. No.: 19/214,387

(22) Filed: Jun. 4, 2025

Related U.S. Application Data
(63) Continuation-in-part of application No. 17/832,213, filed on Jun. 3, 2022.
(60) Provisional application No. 63/196,759, filed on Jun. 4, 2021.

(51) Int. Cl. G06Q 40/02; A61L 2/10; G06Q 10/20

(52) U.S. Cl. CPC G06Q 40/02; G06Q 10/20; A61L 2/10; A61L 2202/11; A61L 2202/23

(57) ABSTRACT

Performing, with a controller of a drive up banking system, a preventative maintenance module for a control system that includes any of an entrance control system, a financial equipment system, a drive-thru audio system, or a pneumatic control system. The preventative maintenance module includes receiving, with the communication system, a current equipment identifier from equipment that includes a universal board. The equipment is associated with any of the entrance control system, the financial equipment system, the drive-thru audio system, or the pneumatic control system. Determining, with the controller, whether the current equipment identifier is within a performance threshold of a historical performance identifier of the equipment. Displaying, in response to the current equipment identifier being within the performance threshold, the current equipment identifier on a user interface. Determining, in response to the current equipment identifier not being within the performance threshold, whether the current equipment identifier is an informational event.

### CROSS-REFERENCE TO RELATED APPLICATION

The present application is a continuation-in-part of U.S. Nonprovisional application Ser. No. 17/832,213 filed on Jun. 3, 2022, which claims the benefit of U.S. Provisional Application No. 63/196,759 filed on Jun. 4, 2021, wherein both applications are incorporated herein by reference.

### FIELD OF THE DISCLOSURE

The present disclosure generally relates to an ultraviolet light system for disinfecting a carrier in a drive-through banking system, and more particularly, but not exclusively to disinfecting the carrier that can travel between teller and customer stations of the drive-through banking system under certain conditions.

### BACKGROUND

Drive-up banking allows customers the convenience of being able to perform banking transactions with tellers inside the bank without having to leave their automobiles or enter the bank building. A carrier is positioned within a drive-through banking tube that travels between a teller station inside the bank and a customer station that is exterior of the bank in which the customer's automobile or customer is positioned adjacent to. The customer and banker exchange money, coins, deposit slips, identification, and other items necessary to complete a transaction wherein these items are transported in the carrier that travels between the customer and the teller stations. The carrier in the drive-through banking system is typically designed to transport up to 25 pounds of weight between the customer and the teller stations.

One problem with this system is that these carriers are frequently contaminated and dirty because many people handle these carriers, and these carriers transport money and coins which are very dirty materials. As the dirty money and coins are transported within the carrier, the carrier becomes a repository for all the various germs, bacteria, viruses, and dirt on the money and coins as well as from the individuals who handle these items may be carrying. As a result, the carriers can be a potential transmitter of disease.

It is known to remove carriers periodically from the drive-through banking system to wash and clean the carriers. While the carrier is removed, the drive-through banking system is not operable which means that customers cannot be serviced for some period of time until the carrier is returned and assembled with the drive-through banking system. After a dirty carrier is removed, it must be cleaned which is typically done off-site from the bank with a cleaning solution and then the wet carrier is dried. The clean carrier is then returned to the drive-through banking system at a later date. Alternatively, a replacement carrier can be placed in the drive-through banking system while the dirty carrier is being cleaned. However, while the dirty carriers are being cleaned the replacement carrier then becomes dirty with germs, bacteria, viruses, and dirt. Moreover, it is costly to service the carriers by one or more technicians that must retrieve, clean, dry, and return the carriers. Thus, it would be highly desirable if the drive up banking carriers could be regularly sanitized to reduce their ability to transmit bacteria and other diseases without removal of the carrier from the drive-through banking system or interruption of banking services to bank customers. It is also highly desirable to sanitize the carriers after every transaction or as desired by customers and/or bankers to reduce the spread of germs, bacteria, and viruses.

Thus, there remains a significant unmet need for the unique apparatuses, methods, systems and techniques disclosed herein.

### BRIEF SUMMARY OF THE DISCLOSURE

Unique apparatuses, methods, and systems are disclosed for a light kit assembly that is installed with a teller station or a customer station of a drive-through banking system or a retrofit light kit assembly that is installed with a previously built drive-through banking system. These light kit assemblies are configured to provide automated or manual disinfection and/or sanitization of outside surfaces of carriers that are installed in the drive-through banking system to keep tellers, associates, and customers safe such that the carriers can be disinfected at any time during non-operation of the carrier or while the carrier is not moving. Carriers are configured to hold the goods, cash, coins, banking documents, and typically can hold up to about 25 pounds of weight. The carrier travels in a bank shoot between an operator's unit which is typically at the teller station inside a bank building and a customer's unit which is typically exterior to the bank building. The light kit assembly includes one or more light sources such as UV lamps, UVC lamps, and/or UVC LED strips attached to one or more mounting plates. The light kit assembly is attached to side panels of a carrier pocket of either the customer station or the teller station that receives the carrier therein. In one form, the light kit assembly is attached to the customer station and sanitizes the carrier pocket within the customer station. In another form, the light kit assembly is assembled as retrofit wherein the light kit assembly is installed in a carrier pocket assembly that is then installed into an existing teller or customer station by simply removing an outer cover panel of the existing teller or customer station, removing the old carrier pocket, and inserting the retrofit light kit assembly/carrier pocket assembly.

The light sources on the mounting plates are positioned to emit UV wavelengths and/or radiation onto the carrier and the carrier pocket to sanitize the carrier and carrier pocket by exposing both to ionized and/or non-ionized radiation for a time period effective for inactivating the bacteria or viruses that may be present on the outer surface of the carrier and carrier pocket. Non-ionized radiation includes UV light. The unique shape of the mounting plates with unique placement of the light sources attached to the mounting plates and unique placement of two of the mounting plates on opposite side panels of the carrier pocket emit UV light that covers 360 degrees around the carrier to sanitize the exterior surface area of the carrier and the air within the carrier pocket.

When the drive-up lane is open and in a normal operation mode, the light source is turned OFF. Normal operation mode indicates that the carrier is operable to move between the customer station and the teller station and the carrier is accessible for use by customers or bankers. When sanitization of the carrier is desired, the carrier is returned or positioned in the carrier pocket of the customer unit and the drive-up lane is activated to the night or sanitizing mode of operation. It is contemplated that either the teller at the teller station or the customer at the customer station can manually activate the sanitizing mode. In one form, the night or sanitizing lock button on the teller unit is turned ON manually to activate the sanitizing mode. In another form, after a transaction is complete such that the carrier has traveled between the customer station and the teller station or the carrier has been used by a person, then the sanitizing mode of operation is automatically activated. In the sanitizing mode of operation, the carrier is in the carrier pocket of the customer unit, the customer unit door is closed, and thereafter the light sources are turned ON. Optionally an indicator light positioned on an outer face of the customer unit is turned ON. This indicator light maybe a designated color when illuminated to indicate that the sanitizing process has started. For example, the designated color can be blue, red, or green. The customer unit door of the carrier pocket will close before the light source is turned ON to avoid exposing any person to the UV wavelengths and/or radiation. The light source will remain turned ON for a set time period to sanitize the carrier and the carrier pocket. In one form, the set time period is about 3 minutes however a longer or shorter set time period may be provided to adequately sanitize the carrier and the carrier pocket. In another form, the set time period is about 45 seconds. When sanitization of the carrier is complete, the carrier will then be sent to the operator or teller unit and the lane will return to normal operating mode. Alternatively, upon completion of sanitization or interruption of sanitization, the carrier can remain in the customer unit. If desired, sanitizing can be interrupted or stopped by turning OFF the night or sanitizing mode switch. The set time period will then be interrupted or stopped and the light source will be turned OFF and thereafter the customer unit door will be operable to open and expose the carrier in the carrier pocket.

Beneficially, the light kit assembly is used to disinfect the carrier between transactions by different persons to kill germs and viruses without removal of the carrier from the drive-through banking system. Therefore, there is less down time of the drive-through banking system because the carrier does not need to be removed for cleaning. Moreover, the carrier and carrier pocket can be disinfected after every use which also leads to improved cleanliness for customers and employees. The light kit assembly reduces transmission of bacteria, viruses and other pathogens via the carrier. In one form, for a set time period of 3 minutes the light source including UVC germicidal LED strips emit 275 nm wavelengths to achieve 99.9% sanitizing effect of the carrier, and at approximately 45 seconds the light kit assembly can sanitize approximately 85% of the carrier's outer surface. Beneficially, there is about 80% energy savings with a light source that includes LED over other forms. Beneficially, there are about 10,000 service hours or 2½ year lane operation with a light source that includes one or more LED's.

Beneficially installation of universal boards with the light kit assembly and drive-through banking system is quick. For example, the customer unit cabling is simplified, a single 16-position rotary switch identifies the lane number, and the universal boards do not have high-voltage. There is no timing setup required because carrier detection sensors will be on all universal board enabled pneumatic systems.

Other benefits of assembly of universal boards with the light kit assembly and drive-through banking system include enabling a user to determine situations in which an error may occur but the safety and operation of the drive-through banking system is not affected thereby keeping the drive-through banking system running when such an error occurs. One or more steps are implemented to determine if the error is an informational event, an atypical device event, an error device event, or a detrimental device event. As such the error is quantified to diagnose problems much quicker and determine which errors require a technician to resolve and which errors do not affect the safety and operation of the drive-through banking system. The errors can be sent automatically to users, including users that are remote, to diagnosis the error so the proper parts can be brought to the site thereby reducing trips for technicians and improving up-time or running time of the drive-through banking system. Diagnosis of the errors also reduces site trips and wasted time by technicians on events that do not interfere with operation of the drive-through banking system. Other benefits include being able to diagnose problems and order replacement parts based on the error, before visiting the site that includes the actual equipment, saving both time and trips to and from the site.

Other benefits of assembly of universal boards with the light kit assembly and drive-through banking system include preventative maintenance alerts to notify a user or technician when an event is within a threshold of an average lifespan of a component. The predictive maintenance tracks and records the performance of a component to predict if the component is close to the end of its lifespan before the component actually breaks down.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method that includes performing, with a controller of a drive up banking system, a preventative maintenance module for a control system that includes one or more of an entrance control system, a financial equipment system, a drive-thru audio system, or a pneumatic control system: receiving, with a communication system, a current equipment identifier from equipment that includes a universal board, where the equipment is associated with any of the entrance control system, the financial equipment system, the drive-thru audio system, or the pneumatic control system; determining, with the controller, whether the current equipment identifier is within a performance threshold of a historical performance identifier of the equipment; displaying, in response to the current equipment identifier being within the performance threshold, the current equipment identifier on a user interface; determining, in response to the current equipment identifier not being within the performance threshold, whether the current equipment identifier is an informational event. Other embodiments of this aspect include corresponding computer systems, apparatus, and computer programs recorded on one or more computer storage devices, each configured to perform the actions of the methods.

Implementations may include one or more of the following features. The method where in response to the current equipment identifier being within the performance threshold having been satisfied, may include determining a type of maintenance required for the equipment. The historical performance identifier includes an average life-expectancy of the equipment. In response to the current equipment identifier being the informational device event having been satisfied, may include continuing operation of the equipment. In response to the current equipment identifier not being the informational device event having been satisfied, determining whether the current equipment identifier is an atypical device event; in response to the current equipment identifier being the atypical device event having been satisfied, performing a clearing workflow of a carrier between a customer unit and a teller unit of the drive up banking system; and determining whether the clearing workflow was successful, and in response to the clearing workflow being successful changing the current equipment identifier to the informational device event. In response to the current equipment identifier not being the atypical device event having been satisfied, determining whether the current equipment identifier is an error device event; in response to the current equipment identifier being the error device event having been satisfied, performing a clearing workflow of a carrier between a customer unit and a teller unit of the drive up banking system; and determining whether the clearing workflow was successful, and in response to the clearing workflow being successful changing the current equipment identifier to the informational device event. In response to the current equipment identifier not being the error device event having been satisfied, determining whether the current equipment identifier is a detrimental device event; and in response to the current equipment identifier being the detrimental device event having been satisfied, displaying the current equipment identifier on the user interface. The detrimental device event corresponds to a failure in the equipment. In response to the current equipment identifier being the detrimental device event having been satisfied, determining a type of maintenance needed on the equipment.

Implementations may include one or more of the following features. The performing the clearing workflow includes: moving, via the controller, the carrier to the teller unit; in response to moving the carrier to the teller unit, determining whether the carrier is physically located at the teller unit; and where in response to the carrier not being physically located at the teller unit being satisfied, the current equipment identifier is the atypical device event. The performing the clearing workflow includes: where in response to the carrier being physically located at the teller unit being satisfied, then moving, via the controller, the carrier from the teller unit to the customer unit; in response to moving the carrier to the customer unit, determining whether the carrier is physically located at the customer unit; and where in response to the carrier not being physically located at the customer unit being satisfied, the current equipment identifier is the atypical device event. The performing the clearing workflow includes: where in response to the carrier being physically located at the customer unit being satisfied, then recalling, via the controller, the carrier from the customer unit to the teller unit; in response to recalling the carrier to the teller unit, determining whether the carrier is physically located at the teller unit; and where in response to the carrier not being physically located at the teller unit being satisfied, the current equipment identifier is the atypical device event. The performing the clearing workflow includes: where in response to recalling the carrier such that the carrier is physically located at the teller unit being satisfied, then opening, via the controller, a door of either the customer unit or the teller unit. The atypical device event includes determining that the carrier was not detected at the teller unit when the carrier was sent to the teller unit or the carrier was not detected at the customer unit when the carrier was sent to the customer unit. The detection events correspond to either the carrier not being detected at the teller unit or the customer unit; determining whether the number of tracked detection events is greater than a maximum number of tracked detection events; and in response to the number of tracked detection events being greater than the maximum number not being satisfied, changing the current equipment identifier to the informational device event. The determining whether the current equipment identifier is an atypical device event includes: moving, via the controller, the carrier to either the teller unit or the customer unit; in response to moving the carrier, tracking a number of detection events where the detection events correspond to either the carrier not being detected at the teller unit or the customer unit as instructed by the moving step; determining whether the number of tracked detection events is greater than a maximum number of tracked detection events; and in response to the number of tracked detection events being greater than the maximum number being satisfied, changing the status of the current equipment identifier to an atypical device event. The maximum number is 10. The informational device event includes a current status of any of the entrance control system, the financial equipment system, the drive-thru audio system, and the pneumatic control system. The informational device event includes a current status of a customer unit or a teller unit associated with the drive up banking system. Implementations of the described techniques may include hardware, a method or process, or computer software on a computer-accessible medium.

One general aspect includes the method including discontinuing operation of the equipment in response to the current equipment identifier being the detrimental device event having been satisfied. Other embodiments of this aspect include corresponding computer systems, apparatus, and computer programs recorded on one or more computer storage devices, each configured to perform the actions of the methods.

This summary is provided to introduce a selection of concepts that are further described below in the illustrative embodiments. This summary is not intended to identify key or essential features of the claimed subject matter, nor is it intended to be used as an aid in limiting the scope of the claimed subject matter. Further embodiments, forms, objects, features, advantages, aspects, and benefits shall become apparent from the following description and drawings.

### BRIEF DESCRIPTION OF THE DRAWINGS

The description herein makes reference to the accompanying drawings wherein like reference numerals refer to like parts throughout the several views, and wherein:

FIG. 1 is a perspective view of a light kit assembly positioned in a customer unit with a carrier.

FIG. 2 is a partial front view of the of the FIG. 1 embodiment.

FIG. 3 is a perspective view of a teller unit that is operably connected to the FIG. 1 embodiment.

FIG. 4 is a front exploded view of the light kit assembly of the FIG. 1 embodiment in a dis-assembled configuration.

FIG. 5 is a front perspective view of the light kit assembly of the FIG. 4 embodiment in an assembled configuration.

FIG. 6 is front view of two of light sources for use with the light kit assembly of the FIG. 1 embodiment.

FIG. 7 is a front view of the two light sources of the FIG. 4 embodiment in a dis-assembled configuration.

FIG. 8 is a front perspective view of the mounting plates of the FIG. 4 embodiment.

FIG. 9 is a partial perspective view of a fastener and washer of the FIG. 4 embodiment assembled with one of the light sources.

FIG. 10 is a rear view of the FIG. 5 embodiment.

FIG. 11 is a front view of one embodiment of a wire harness input that can be assembled with the FIG. 1 embodiment.

FIG. 12 is a second illustration of the wire harness input of the FIG. 11 embodiment.

FIGS. 13 and 14 are front views of one embodiment of a wire harness output that can be assembled with the FIG. 1 embodiment.

FIG. 15a is side view of a colored indicator light of the FIG. 1 embodiment.

FIG. 15b is an end view of the colored indicator light of the FIG. 15a embodiment.

FIGS. 16-21 are front views of a power supply assembly of the FIG. 1 embodiment.

FIG. 22 is a front view of a panel of the carrier pocket of the customer unit.

FIG. 23 is a front view of the light kit assembly attached to the panel of the carrier pocket of the customer unit of the FIG. 22 embodiment.

FIG. 24 is a front view of a power supply assembly for assembly to an outside face of the panel of the carrier pocket of the customer unit of the FIG. 22 embodiment.

FIG. 25 is a front view of a split grommet.

FIG. 26 is a front view of the split grommet of FIG. 25 positioned into a wire access hole of the FIG. 22 embodiment.

FIG. 27 is a front view of an optional indicator light assembled with the customer unit.

FIG. 28 is a front view of the optional indicator light of FIG. 27 being assembled with the customer unit.

FIG. 29 is a front view of the optional indicator light of FIG. 27 being assembled with the customer unit.

FIG. 30 is a front view of a second embodiment of a light kit assembly assembled with a replacement carrier pocket.

FIG. 31 is a partial perspective view of the light kit assembly assembled with the replacement carrier pocket installed in the customer unit of the FIG. 30 embodiment.

FIG. 32 is an illustrative embodiment of a control system configured to interact with one or more controllers.

FIG. 33 is an illustrative embodiment of a plurality of universal boards configured for assembly with the control system of FIG. 32 embodiment.

FIG. 34 is an illustrative embodiment of a plurality of equipment identifiers.

FIG. 35 is an illustrative embodiment of a plurality of device events.

FIG. 36 is exemplary embodiment of a main controller universal board for assembly with the control system of FIG. 32 embodiment.

FIG. 37 is exemplary embodiment of an expansion universal board for assembly with the control system of FIG. 32 embodiment.

FIG. 38 is exemplary embodiment of a motor control board for assembly with the control system of FIG. 32 embodiment.

FIG. 39 is exemplary embodiment of an audio expansion board for assembly with the control system of FIG. 32 embodiment.

FIG. 40 is an exemplary embodiment of a user interface for assembly with the control system of FIG. 32 embodiment.

FIG. 41 is an exemplary embodiment of a queuing screen displayed on the user interface of the FIG. 40 embodiment.

FIG. 42 is an exemplary embodiment of a setup screen displayed on the user interface of the FIG. 40 embodiment.

FIG. 43 is an exemplary embodiment of a service panel displayed on the user interface of the FIG. 40 embodiment.

FIG. 44 is an exemplary embodiment of a remote dashboard displayed on the user interface of the FIG. 40 embodiment.

FIG. 45 is an exemplary embodiment of a clearing workflow for operation of a carrier between a customer unit and a teller unit.

FIG. 46 is one embodiment of a preventative maintenance module operable with the control system of FIG. 32 embodiment.

FIG. 47 is a front view of an illustrative embodiment of a main controller universal board for assembly with the control system of FIG. 32 embodiment.

FIG. 48 is a rear view of the main controller universal board of FIG. 47.

### DESCRIPTION OF ILLUSTRATIVE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, any alterations and further modifications in the illustrated embodiments, and any further applications of the principles of the invention as illustrated therein as would normally occur to one skilled in the art to which the invention relates are contemplated herein.

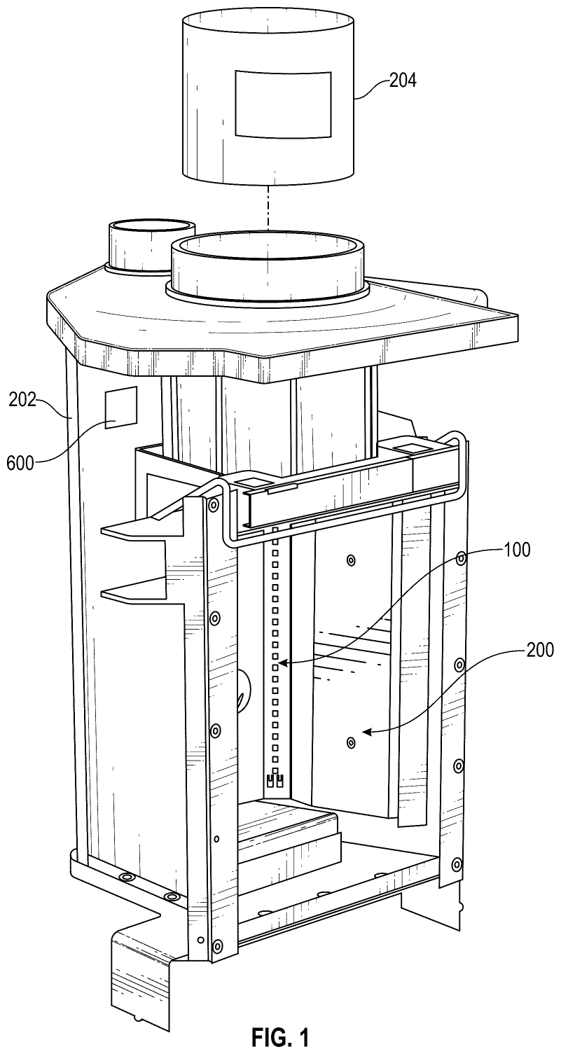

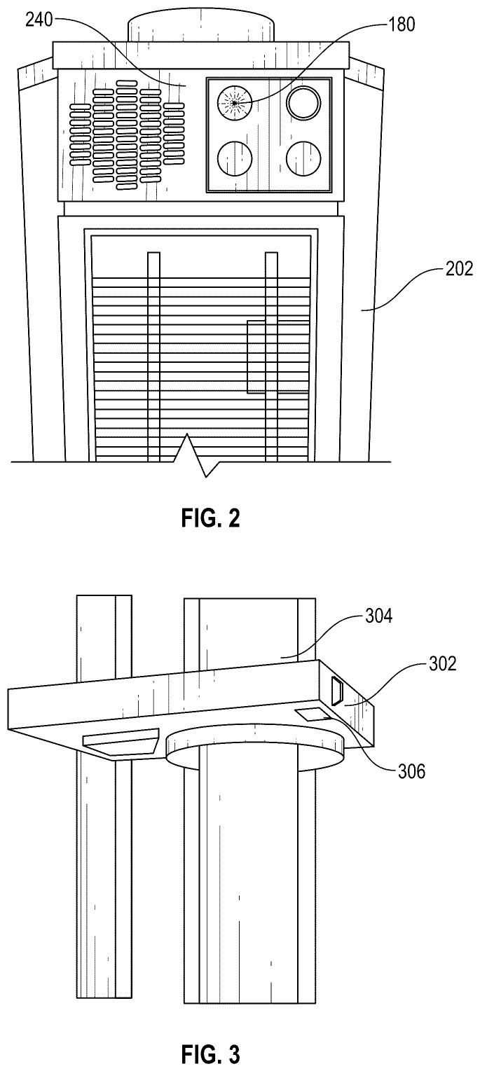

Referring to FIGS. 1 and 2 is a first embodiment of a light kit assembly 100 assembled within a carrier pocket 200 of a customer unit 202 that receives a carrier or cylinder 204 therein that is configured to hold goods, money, and other banking items. One embodiment of the carrier or cylinder 204 is illustrated above the customer unit 202, however the customer unit 202 is assembled with a bank shoot 304 (FIG. 3) through which the carrier 204 travels between the customer unit 202 and a teller station 302. Other forms of the carrier 204 can be used to travel in the bank shoot 304 between the customer unit 202 and the teller station 302. The light kit assembly 100 provides an automated or manual disinfection or sanitization process at a drive-thru banking location to keep persons safe from germs or diseases during the transfer of cash and goods in the carrier 204 that travels between the customer station or unit 202 and the teller station or unit 302 as illustrated in FIG. 3. The teller unit 302 is typically inside a banking facility or building. The teller unit 302 includes a night or sanitizing lock switch 306 that enables someone to manually operate the light kit assembly 100. The customer unit 202 is connected to the teller unit 302 via a bank shoot 304 (partially illustrated in FIG. 3) which is a drive-through banking transit tube that holds the carrier 204 as it travels between the customer unit 202 and the teller unit 302.

It shall be appreciated that the illustrated configuration and components of the light kit assembly 100 are one example embodiment, and that the disclosure contemplates that a variety of different light kit assemblies 100 and associated components thereof may be utilized. It is also contemplated that the light kit assembly 100 can be positioned in the teller unit 302, the customer unit 202, or another location in the drive-through banking transit tube. The light kit assembly 100 can also be a retrofit assembly that is positioned in the teller unit 302 or the customer unit 202 wherein the retrofit assembly may include different connections to units 302 or 202.

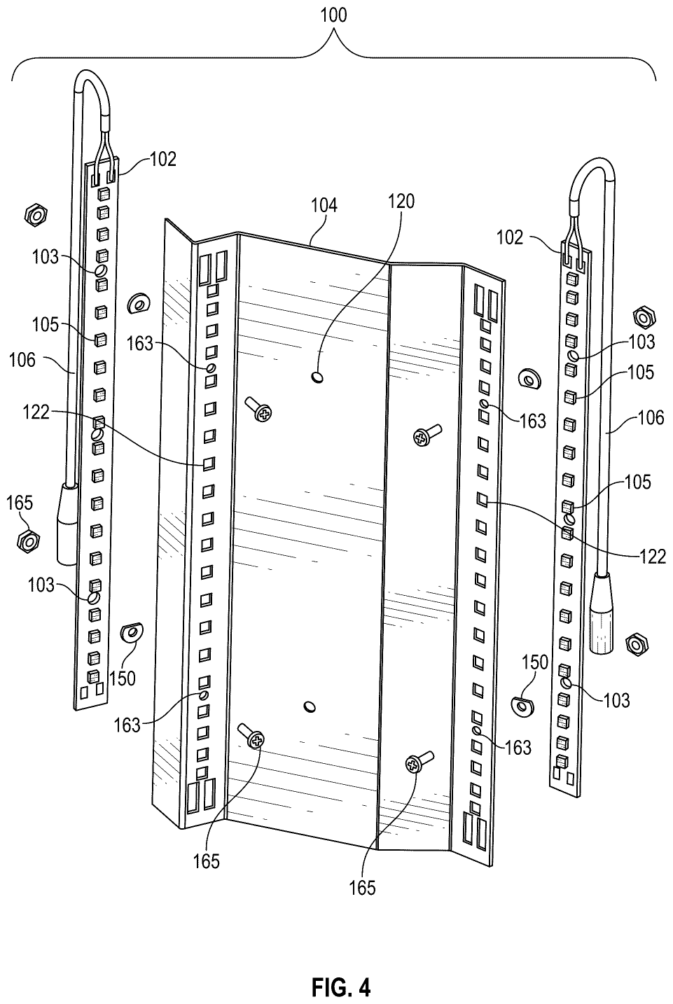

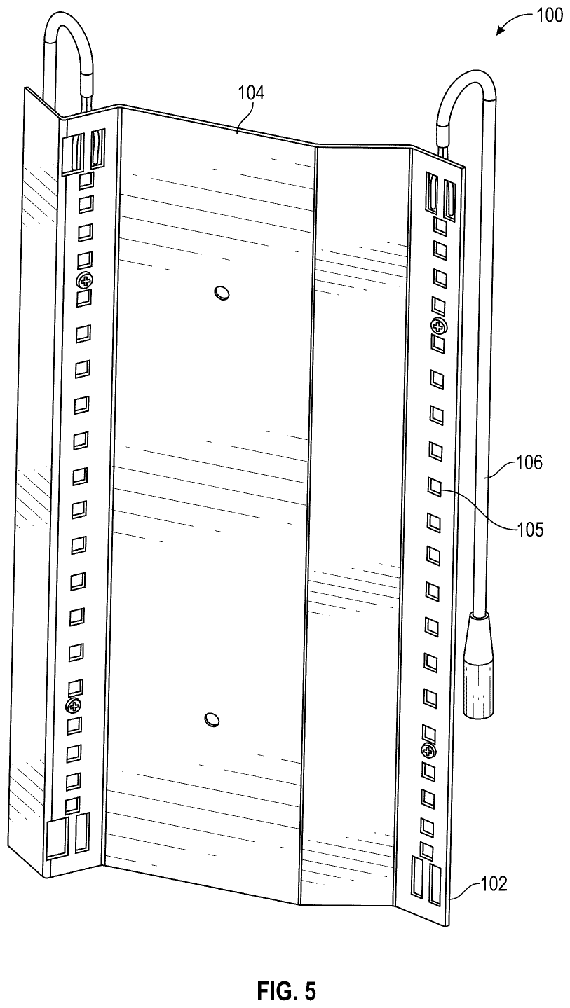

Turning to FIGS. 4 and 5, is one embodiment of the light kit assembly 100. In the illustrated embodiment, the light kit assembly 100 includes two light sources 102 that are attached to a mounting plate 104. In other embodiments, the light kit assembly 100 can include one or more light sources 102.

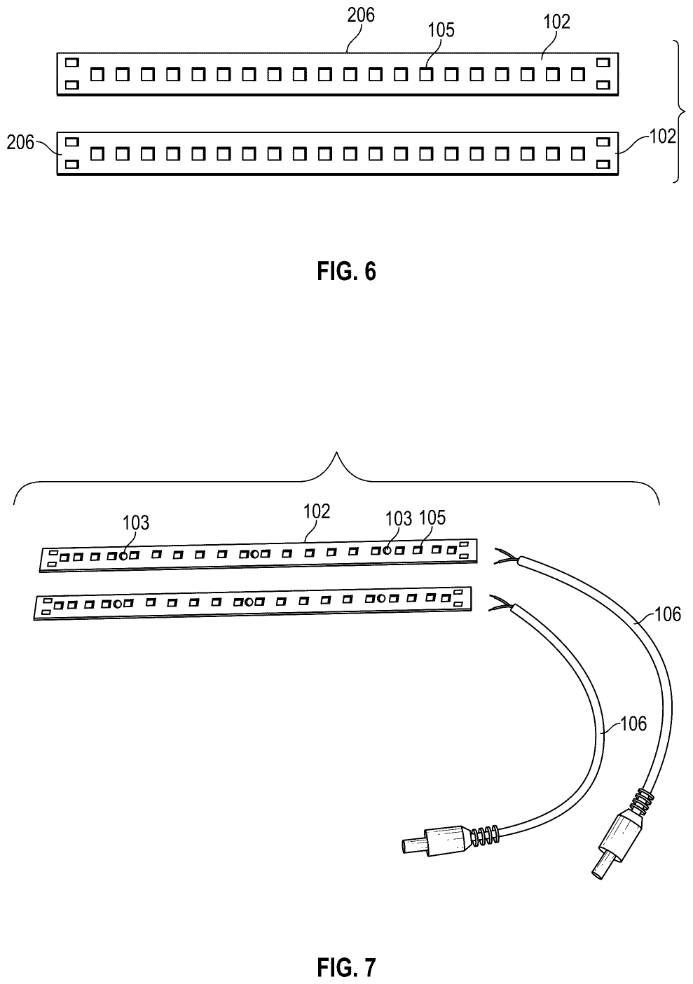

In the illustrated embodiment, the light sources 102 include a plurality of ultraviolet-C “UVC” lamps 105 assembled into UVC LED strips 206 as shown in FIG. 6. In one form, the UVC LED strips 206 emit ultraviolet light having 275 nm wavelength and are approximately 210 mm long and 12 mm thick. UVC radiation is a disinfectant for air, water, and nonporous surfaces. Other configurations or types of UVC LED strips can be used in other embodiments of the light sources 102. UVC radiation reduces the spread of bacteria and viruses and are frequently known as “germicidal” lamps. The light sources 102 configured as UVC LED strips 206 may emit a broad range of UV wavelengths, and may also emit infrared or electromagnetic radiation. Other embodiments of the light sources 102 may include UV lamps 105, or alternatively and/or additionally, mercury lights, Xenon lights, and/or excimer lamps, to name a few examples. It is contemplated that other types of light or energy sources may be used to disinfect or sanitize the carrier and the carrier pocket 200 when assembled with the customer unit 202. The light sources 102 include a plurality of mounting holes 103 for assembly of the light sources 102 to the mounting plates 104 such that the mounting holes 103 align with a corresponding number of mounting holes 163 in the mounting plates 104 wherein a fastener or other connector is inserted through the mounting holes 103 and 163 to attach the light sources 102 to the mounting plates 104 as described in more detail below. The plurality of UVC lamps 105 are arranged and configured to align with a corresponding number of light holes in the mounting plates 104 to assemble the light sources 102 thereto such that the UVC lamps 105 of the light sources 102 emit light through the light holes in the mounting plates 104 as described in more detail below.

Turning to FIG. 7, power cables 106 are illustrated that can be attached to the light sources 102. In one form, the power cables 106 are solder power cables 69-A1P attached to light sources 102. In other embodiments, the power cables 106 may be configured differently.

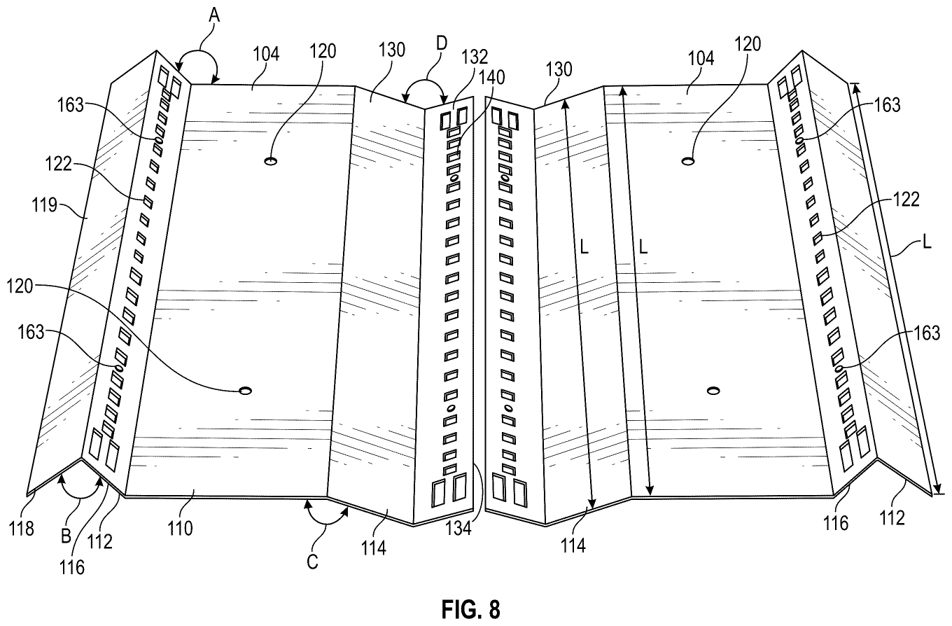

Turning to FIG. 8, the two mounting plates 104 are identical to each other therefore for the sake of brevity only one mounting plate 104 will be described. As illustrated, the first mounting plate 104 is rotated 180 degrees from the second mounting plate 104. In other embodiments, the mounting plates 104 maybe configured differently from each other. The mounting plates 104 can be made of any material, for example, stainless steel, metal, and/or plastic, that can be configured to receive the light sources 102. Each of the mounting plates 104 includes a back wall 110 that extends between a first wing 112 and a second wing 114. Each of the back wall 110 and the first and second wings, 112 and 114, respectively, have a length, L, that approximately corresponds to or is longer than a length of the light sources 102. In other embodiments the length, L, of the back wall 110 may be longer or shorter than the length, L, of the first and second wings, 112 and 114. The back wall 110 includes two openings 120 sized to receive a fastener or an electric cord there through.

The first wing 112 has a bi-folded shape with a first portion 116 adjacent the back wall 110 and extending to a second portion 118 that includes a free edge 119. A first backwall angle A spans between the first portion 116 and the back wall 110. A first wing angle B spans between the first portion 116 and the second portion 118. The first portion 116 includes a plurality of light holes 122 sized to receive the UVC lamps 105 on the light source 102 therein to further assemble and/or mount the light source 102 to the first portion 116 such that one of the light holes 122 is assembled with one of the UVC lamps 105. The second portion 118 does not include any openings or light holes but may in other embodiments. For example, in an alternative embodiment, the second portion 118 may include a plurality of light holes that are sized to receive the UVC lamps 105 on the light source 102 therein to further assemble and/or mount the light source 102 to the second portion 118.

The second wing 114 has a bi-folded shape with a first portion 130 adjacent the back wall 110 and extending to a second portion 132 that includes a free edge 134. The first portion 130 does not include any openings or light holes but may in other embodiments. For example, in an alternative embodiment, the first portion 130 may include a plurality of light holes that are sized to receive the UVC lamps 105 on the light source 102 therein to further assemble and/or mount the light source 102 to the first portion 130. The second portion 132 includes a plurality of light holes 140 sized to receive the UVC lamps 105 on the light source 102 therein to further assemble and/or mount the light source 102 to the second portion 132. A second backwall angle C spans between the first portion 130 and the back wall 110. A second wing angle D spans between the first portion 130 and the second portion 132.

The unique folded shape of the mounting plate 104 and unique placement of two of the mounting plates 104 on opposite side panels of the carrier pocket 200, when the light kit assembly 100 is assembled with the carrier pocket 200, enable the light sources 102 to emit UV light to cover an area of approximately 360 degrees to sanitize all or substantially all of the exterior surface area of the carrier 204 and the air within the carrier pocket 200. This is beneficial because UV light does not penetrate through the exterior surface of the carrier 204 to the interior of the carrier 204. Therefore the unique placement of light sources 102 on the mounting plates 104 and the unique configuration of the mounting plates 104 emit wavelength around the outer surface of the carrier 204 that is highly effective to sanitize the outer or exterior surface area of the carrier 204 that an individual may touch or contact.

To assemble the light sources 102 with the mounting plates 104, the two mounting plates 104 are arranged on a surface in a mirrored position to each other as illustrated in FIG. 8. This arrangement is also beneficial for installation of the mounting plates 104 with the left and right panels of the carrier pocket 200 because the mirrored arrangement of the mounting plates 104 in the carrier pocket 200 enables more of the exterior surface area of the carrier 204 to be sanitized. In other embodiments, the mounting plates 104 are not positioned side by side and not in a mirrored arrangement in either the assembly of the light sources 102 with the mounting plates 104 or the installation of the mounting plates 104 with the carrier pocket 200.

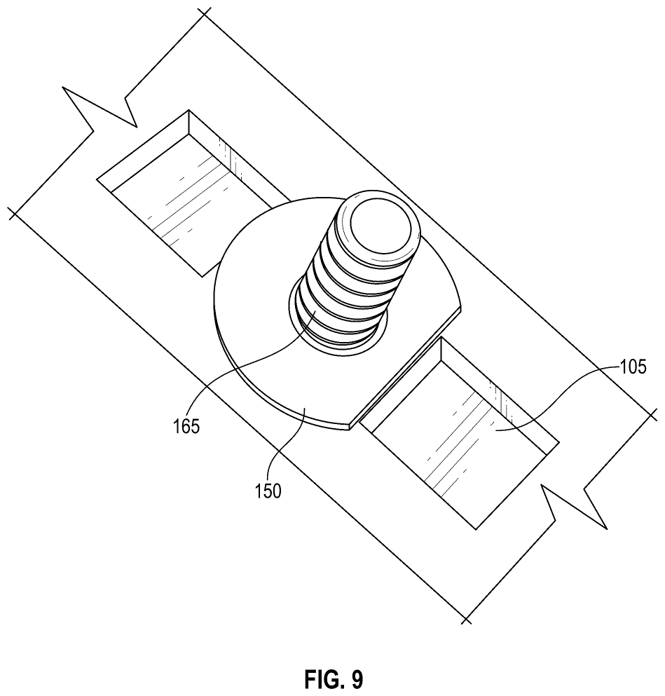

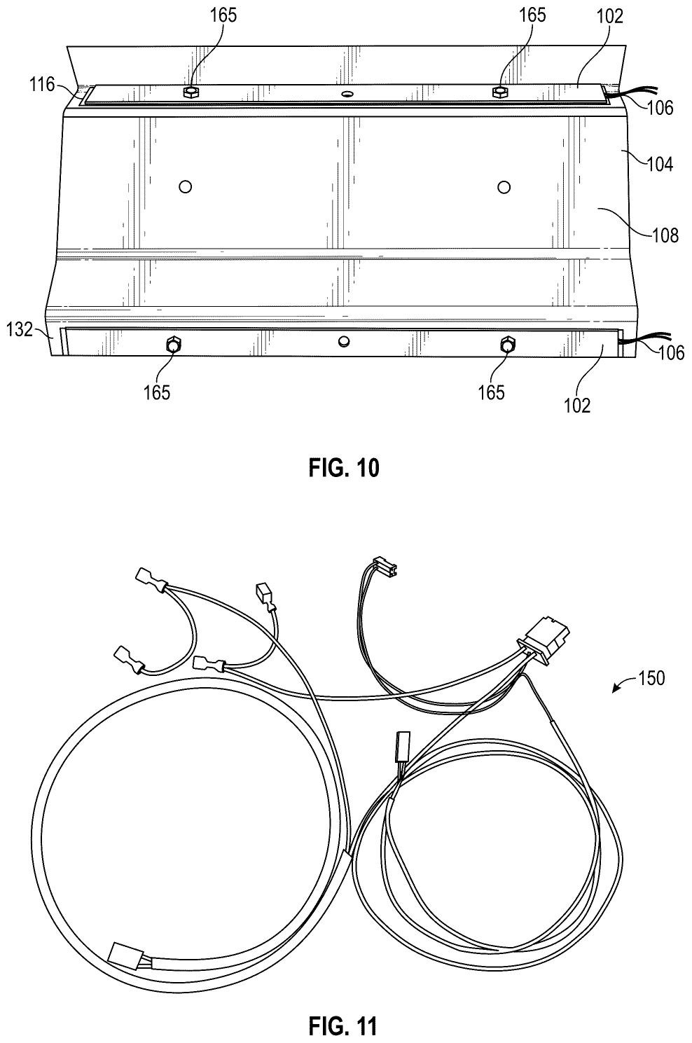

The light sources 102 are assembled to a bottom surface 108 of the mounting plates 104 such that the power cables 106 extend from the bottom surface 108 as illustrated in FIG. 10. The light source 102 is positioned on the bottom surface 108 of the mounting plate 104 such that each of the UVC lamps 105 aligns with one of the plurality of light holes 122 of the first portion 116. Similarly, a second light source 102 is positioned on the bottom surface 108 of the mounting plate 104 such that each of the UVC lamps 105 aligns with one of the plurality of light holes 140 of the second portion 132. In particular, the light sources 102 are mounted with a trimmed nylon washer 150 between the light source 102 and the mounting plate 104 using one of a plurality of fasteners 165 such as, screws and lock nuts, through each of the mounting holes 103 and 163 to attach the light sources 102 to the mounting plates 104. In one embodiment, four round nylon washers 150 are trimmed with a knife, side cutters, or other cutting means, to remove a portion of an edge of the round nylon washers 150. Illustrated in FIG. 9 is the trimmed nylon washer 150 that is assembled with the fastener 165.

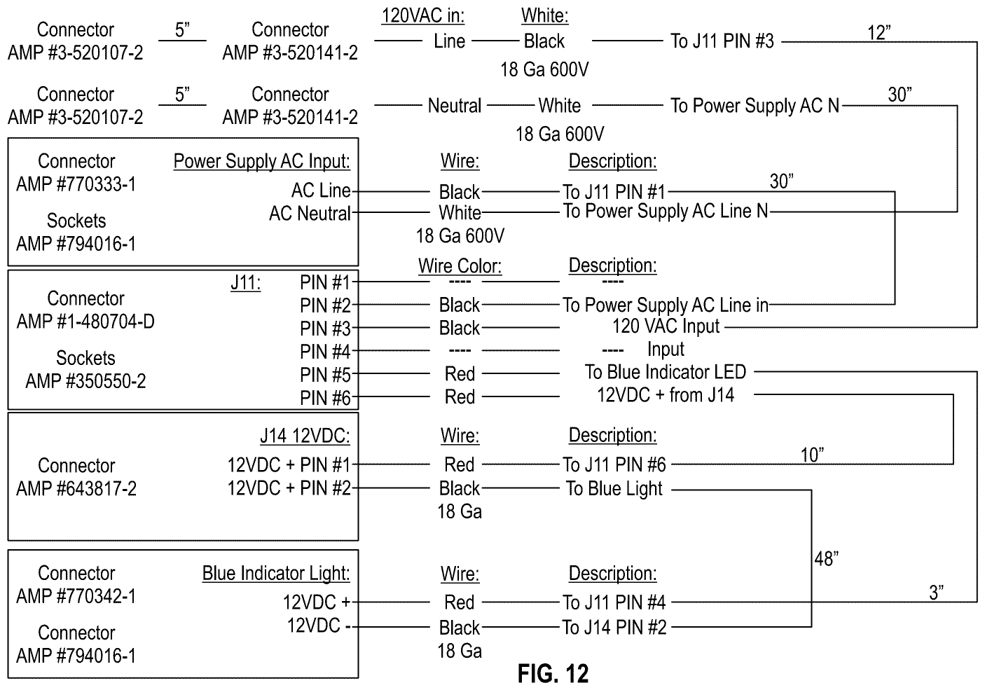

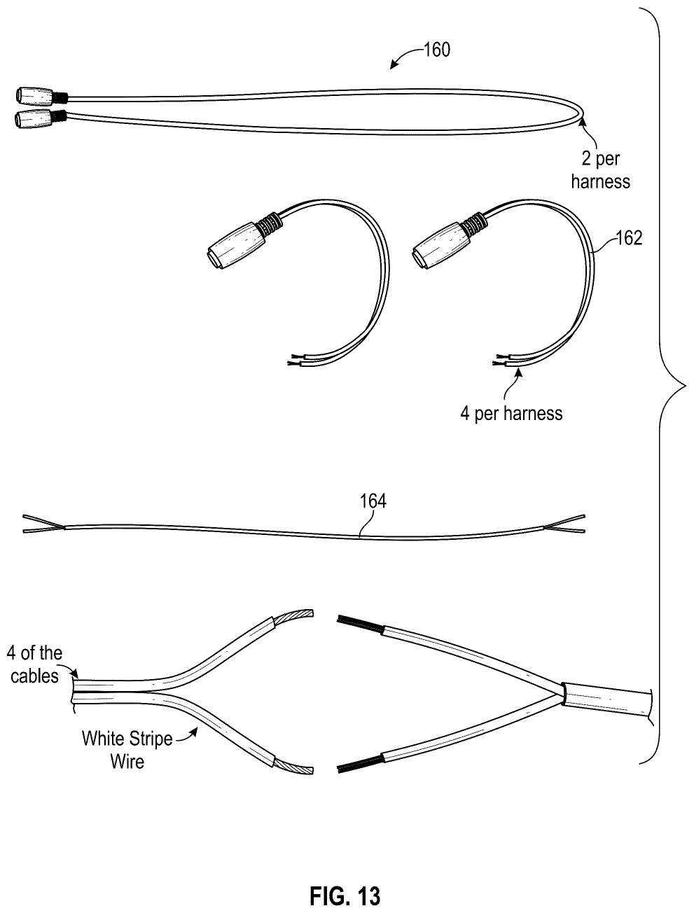

FIGS. 11 and 12 illustrate an embodiment of a wire harness input 150 that is configured for assembly with the light kit assembly 100. In other embodiments, the wire harness input 150 is configured differently.

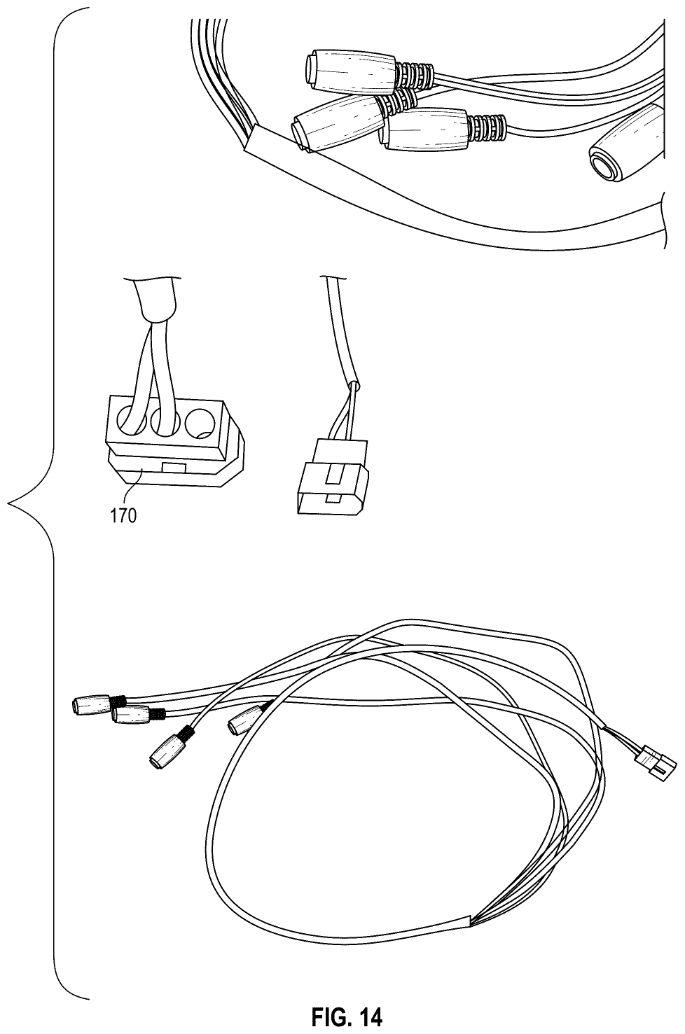

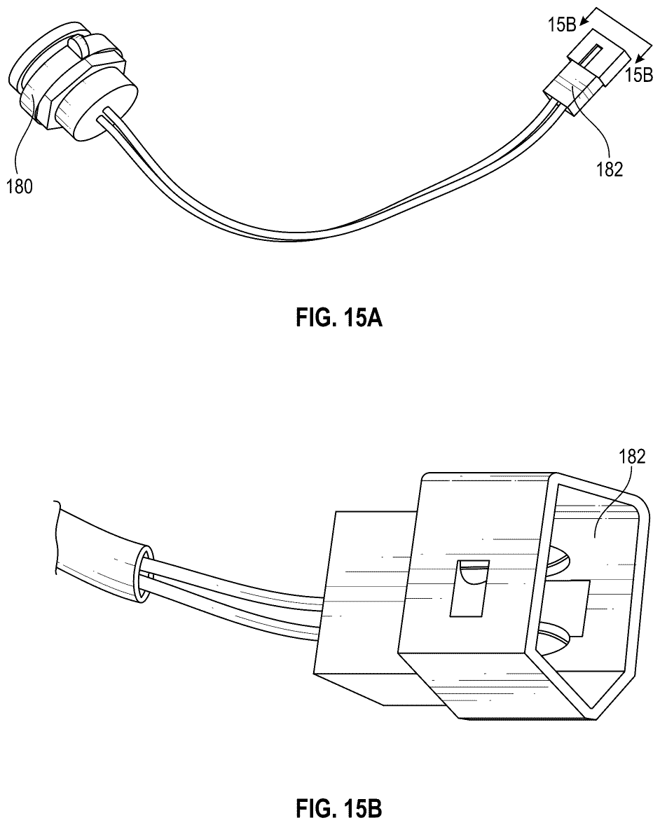

FIGS. 14 and 15 illustrate an embodiment of a wire harness output 160 that is configured for assembly with the light kit assembly 100. In other embodiments, the wire harness output 160 is configured differently. The wire harness output 160 is cut in half to create two cable assemblies 162 wherein the wire conductors of the cable assemblies 162 are split and the ends are stripped approximately ⅜″. The wire harness output 160 includes a conductor cable 164 having a length of approximately 18 inches wherein approximately 1½″ of casing is removed from both ends of the conductor cable 164. The wires on one end of the conductor cable 164 are stripped approximately ⅜″ and the wires on the other end of the conductor cable 164 are stripped approximately ⅛″. Next, the four half cable assemblies 162 are soldered to the two conductor cables 164 and wire the white stripe to red. A first shrink tube (not illustrated) is placed over the red/white stripe connection, then a second shrink tube is placed over both connections. There will be four of these cables. An AMP socket is installed on both of the red and black wires of the cables. Next as illustrated in FIG. 14, an AMP 3-position female connector 170 is installed on the cables such that the red wire is in position #2 and the black wire is in position #3. The #1 position is left empty in the AMP 3-position female connector 170.

FIGS. 15a and 15b illustrate an optional colored indicator light 180 that is configured for assembly with the light kit assembly 100. In one form, the indicator light 180 is a blue LED light however in other embodiments the indicator light 180 may be a different color. The indicator light 180 includes AMP pins to both wires and an AMP 2-position female connector 182 that is attached or connected to both wires. The red wire is located in position #1 and the black wire is located in position #2 of the female connector 182.

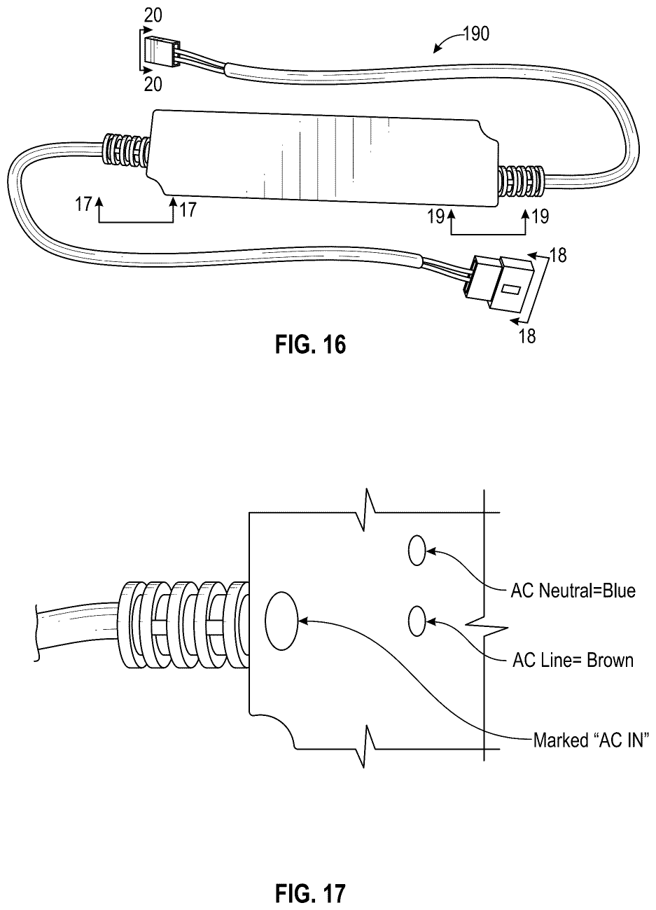

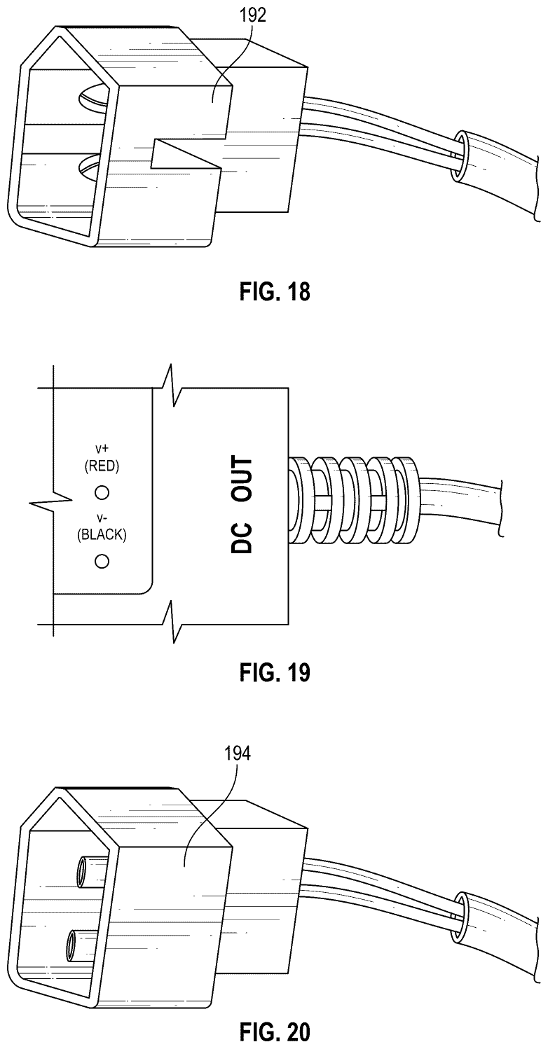

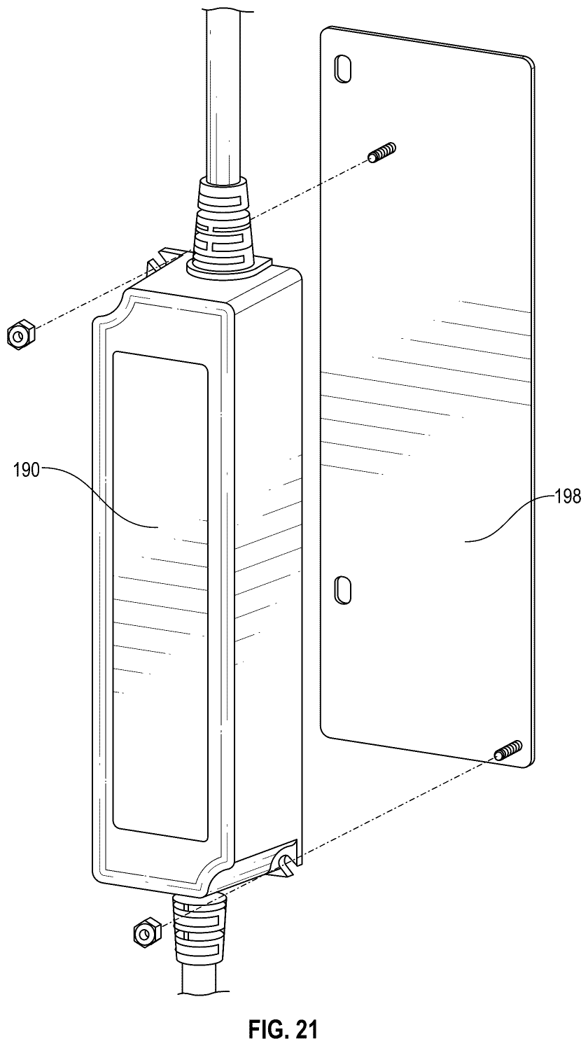

FIGS. 16-20 illustrate a power supply assembly 190 for the light kit assembly 100. In other embodiments, the power supply assembly 190 is configured differently. The power supply assembly 190 includes a power input connection and AMP pins installed to both the blue and brown wires. An AMP 3-position female connector 192 is installed such that the brown wire is placed in position #1, the blue wire is placed in position #3, and the #2 position is left empty. The power supply assembly 190 includes an output power connection. The power supply assembly 190 includes an AMP socket connected to both the red and black wires. An AMP 3-position male connector 194 is connected to the red wire in position #2, the black wire in position #3, and the #1 position is left empty. Illustrated in FIG. 21 is the power supply assembly 190 that is mounted to a mounting plate 198 as shown using a plurality of fasteners such as screws and lock nuts.

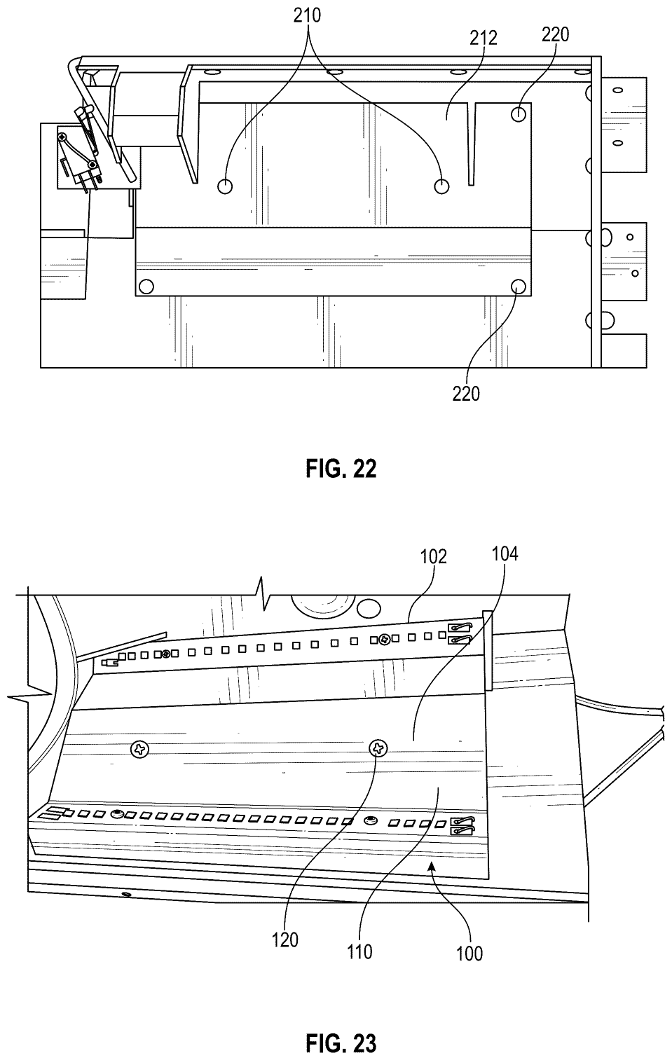

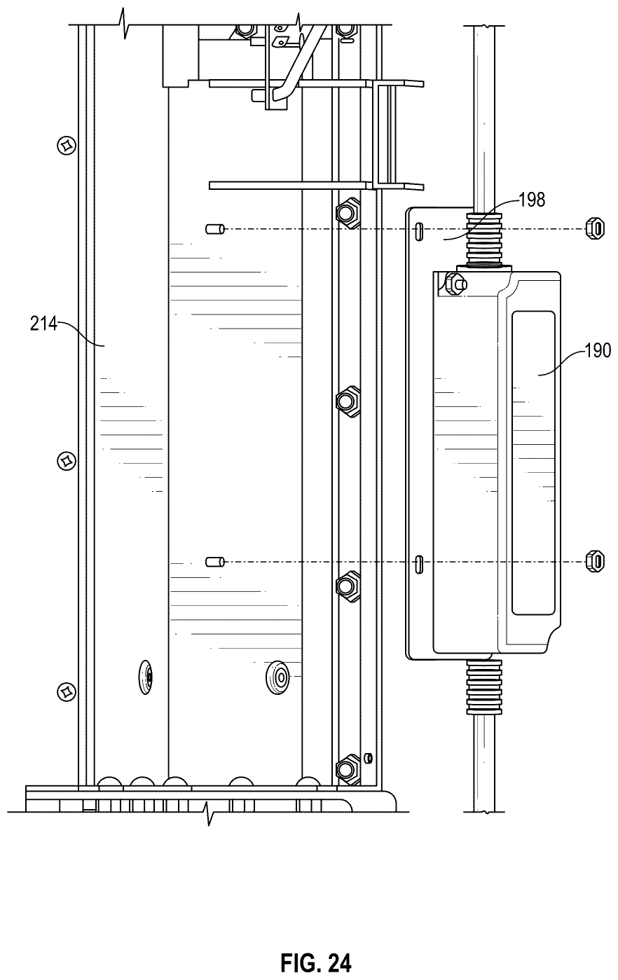

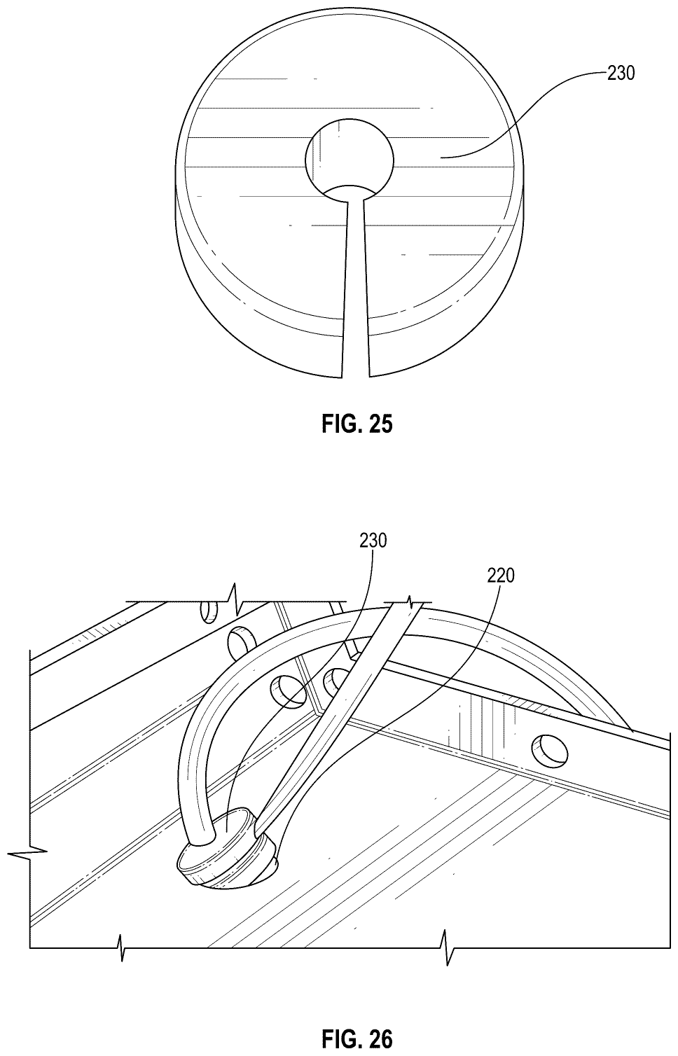

FIGS. 22-26 illustrate the light kit assembly 100 that is mounted to the carrier pocket 200 of the customer unit 202. Two of the light kit assemblies 100 are mounted to the carrier pocket 200, a first one on the left side panel 212 and a second one on a right side panel (not illustrated) of the carrier pocket 200. For the sake of brevity, the attachment of the light kit assembly 100 to a left side panel 212 of the carrier pocket 200 will be described next however the attachment of the light kit assembly 100 to the right side panel of the carrier pocket 200 will be similar. Two mounting plate holes 210 are formed in the left side panel 212 of the carrier pocket 200 of the customer unit 202. The mounting plate holes 210 are sized and positioned to align with the openings 120 in the back wall 110 of the mounting plate 104 when the mounting plate 104 is assembled with the left side panel 212. Two wire access holes 220 are formed in each of the side panels 212 for positioning the power cables 106 through the wire access holes 220 in the left side panel 212. The mounting plate 104 is secured or attached to the left side panel 212 with a fastener, screw, bolt, washer, and/or lock nuts through the mounting plate holes 210 and the openings 120. The power supply assembly 190 is attached to an outside face 214 of the left side panel 212 which is outside of the pocket 200 with a fastener or other means to hold the mounting plate 190 thereon. This process is repeated to mount the second of the light kit assemblies 100 to the right side panel of the carrier pocket 200. In the illustrated embodiment, there is only one of the power supply assembly 190 mounted to the left side panel 212 however in other embodiments there may be a second of the power supply assembly 190 mounted to the right side panel. Optionally as illustrated in FIGS. 25 and 26, a split grommet 230 is positioned over each over each LED wire and positioned into the wire access holes 220.

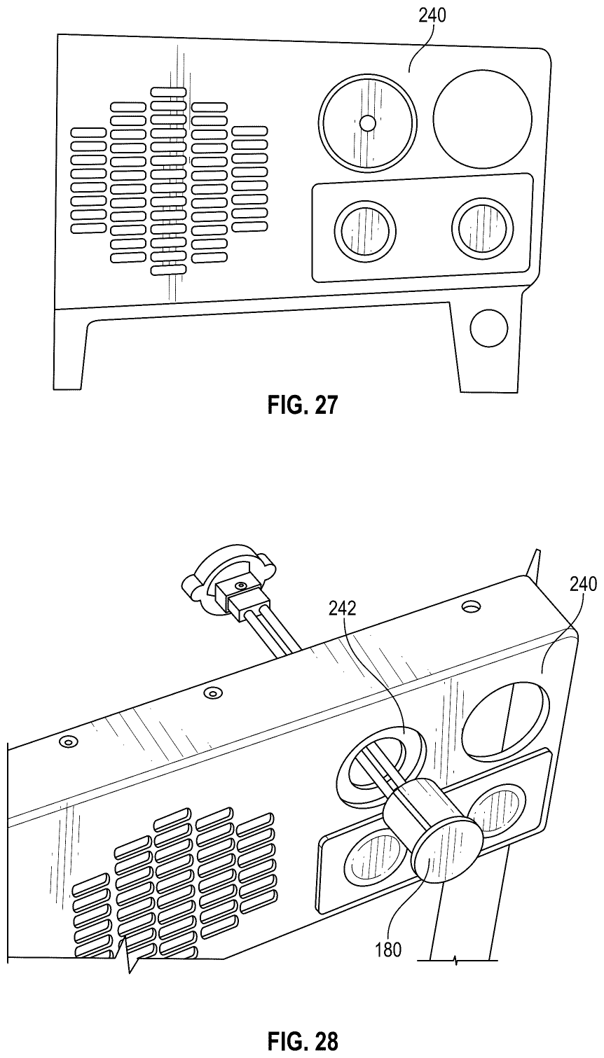

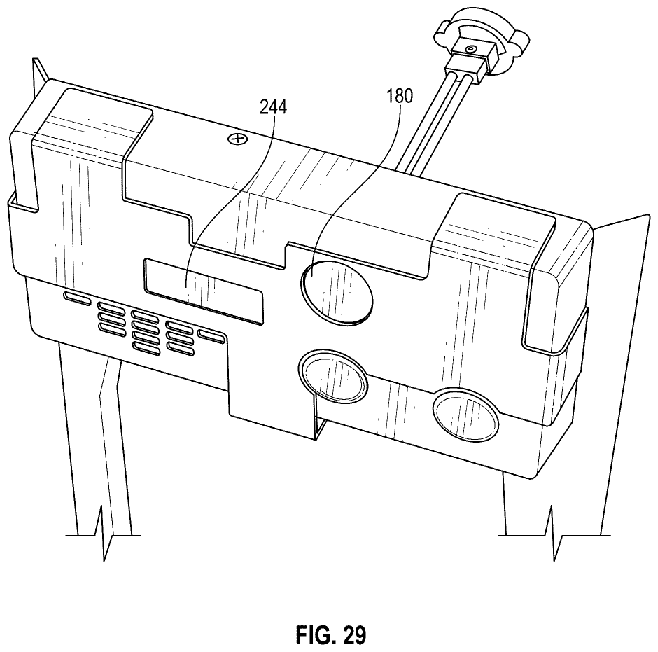

FIGS. 27-29 illustrate the indicator light 180 assembled into an opening 242 in a fascia 240 of the customer unit 202. Optionally, a “Sanitizing” label 244 is attached to the fascia 240. Beneficially, the indicator light 180 alerts a user that the sanitization process of the carrier is being carried out.

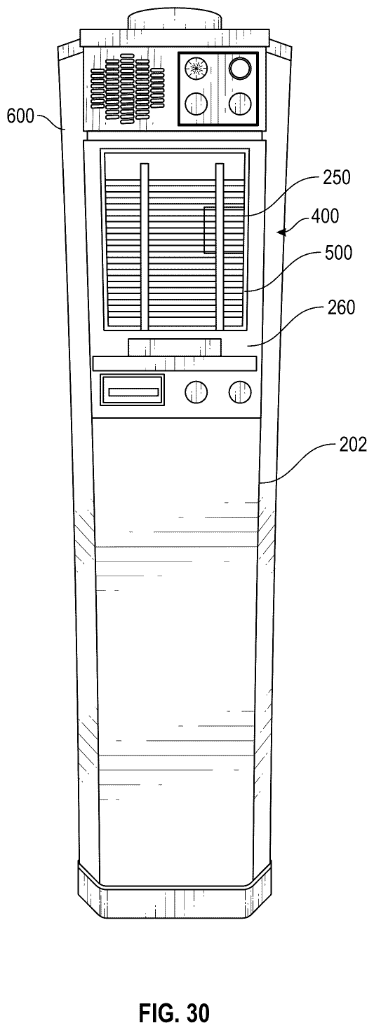

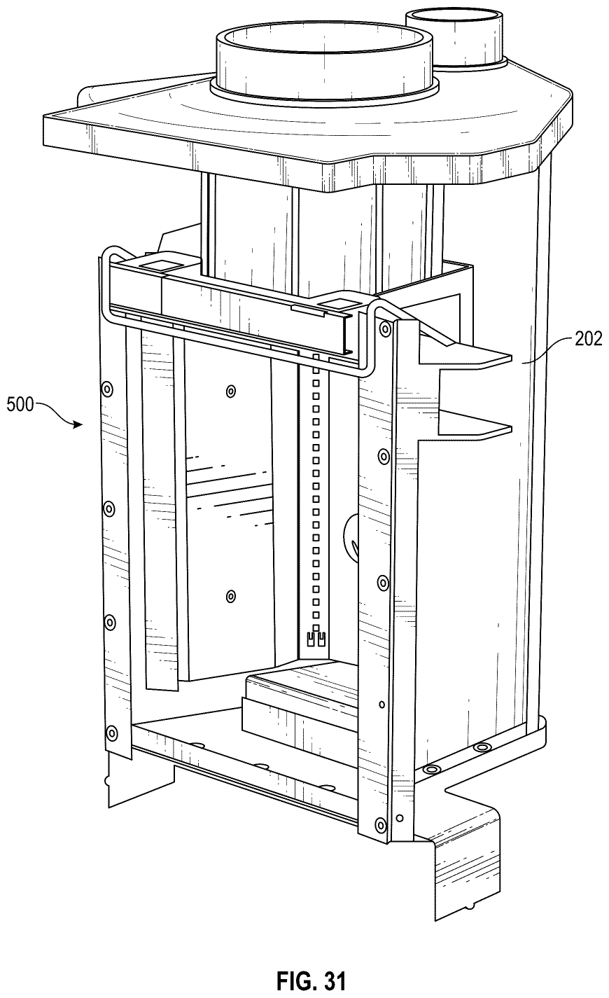

FIG. 30 illustrates a second embodiment of a light kit assembly 400 assembled with a replacement carrier pocket 260 to form a light kit subassembly 500. The light kit subassembly 500 is then installed in a customer unit 202 after removing the existing carrier pocket 200 of the customer unit 202. Also illustrated is the customer unit 202 including a customer unit door 250 in a closed position. The light kit assembly 400 is similar to the light kit assembly 100. As such, the light kit assembly 400 includes one or more light sources 102 that are attached to the mounting plate 104. Illustrated in FIG. 31 is the light kit subassembly 500 installed in the customer unit 202 using one or more fasteners, wherein the customer unit door 250 is removed for clarity.

### IOT/Connectivity Features

In certain embodiments, the light kit assembly 100 is coupled to a controller 600 structured to perform certain operations to control operation of the light source 102, and optionally one or more of the customer unit 202, the teller unit 302, and the carrier 204. In other embodiments, the light kit assembly 400 is coupled to the controller 600. In certain embodiments, the controller 600 forms a portion of a processing subsystem including one or more computing devices having memory, processing, and communication hardware. The controller 600 may be a single device or a distributed device, and the functions of the controller 600 may be performed by hardware or by instructions encoded on computer readable medium. The controller 600 may be included within, partially included within, or completely separated from a teller unit controller or a customer unit controller (not shown). The controller 600 is in communication with any sensor or actuator throughout the light kit assembly 100 or 400, including through direct communication, communication over a datalink, and/or through communication with other controllers or portions of the processing subsystem that provide sensor and/or actuator information to the controller.

In certain embodiments, the controller 600 is described as functionally executing certain operations. The descriptions herein including the controller operations emphasizes the structural independence of the controller 600, and illustrates one grouping of operations and responsibilities of the controller 600. Other groupings that execute similar overall operations are understood within the scope of the present application. Aspects of the controller 600 may be implemented in hardware and/or by a computer executing instructions stored in non-transient memory on one or more computer readable media, and the controller 600 may be distributed across various hardware or computer based components.

Example and non-limiting controller implementation elements include sensors providing any value determined herein, sensors providing any value that is a precursor to a value determined herein, datalink and/or network hardware including communication chips, oscillating crystals, communication links, cables, twisted pair wiring, coaxial wiring, shielded wiring, transmitters, receivers, and/or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), and/or digital control elements.

The drive-up pneumatic system that includes a plurality of universal boards 1500 can also include carrier presence sensors on both the customer unit 202 and the teller unit 302. The carrier presence sensors provide notice of certain operations. For example, when the customer hits the send button on the customer unit 202 to send the carrier 204 to the teller unit 302, the customer door on the customer unit 202 will attempt to close prior to turning on the pneumatic system turbines. If the customer door closed switch is not detected by the carrier presence sensors, such as due to some failure, then the universal board enabled equipment, will turn on the pneumatic turbines and attempt to send the carrier 204 to the teller unit 302. Thereafter, if the carrier sensor at the teller unit 304 detects the carrier 204, indicating that the transfer of the carrier 204 to the teller unit 302 was successful, then it is known that the customer door of the customer unit 202 actually did close even though the customer door closed switch was not detected by the carrier presence sensor. This can be known with certainty because if the customer door did not close then pressure/vacuum would not have been built up in the pneumatic system to send the carrier 204 to the teller unit 302 because the customer door was open. An error message may be sent to the user interface 702 to display this message that the door closed switch/sensor is bad or broken. However, the customer and teller units 202 and 302, respectively, can continue to operate in the meantime or until the door closed switch/sensor is fixed. The customer and teller units 202 and 302, respectively, continue operation during this time and will generate the appropriate warning messages to inform the repair person to go to the site to resolve the issue. Instead of “failing” or closing this drive-thru lane the customer and teller units 202 and 302, respectively, continue to run until the warning can be resolved.

A plurality of universal boards 1500 are illustrated in FIG. 33 wherein each of the universal boards 1500 may be specific for assembly with a particular piece of equipment in the drive up banking system. For example, the plurality of universal boards 1500 can include a main controller universal board 1502 that can be assembled with the controller 600 or the communications system 703, an expansion universal board 1504 that can be assembled with the entrance control system 720 which can include the customer station 202 and the teller station 302, a motor control board 1506 that can be assembled with the pneumatic control system 726, and an audio expansion board 1508 that can be assembled with the drive-thru audio system 724. The plurality of universal boards 1500 are configured to communicate to one or more back end services. In an alternative embodiment, the plurality of universal boards 1500 can include a main controller universal board 2502 that can be assembled with the controller 600 or the communications system 703 and the entrance control system 720. In this embodiment, the expansion universal board is not required.

The listing herein of specific implementation elements is not limiting, and any implementation element for any controller described herein that would be understood by one of skill in the art is contemplated herein. The controllers herein, once the operations are described, are capable of numerous hardware and/or computer based implementations, many of the specific implementations of which involve mechanical steps for one of skill in the art having the benefit of the disclosures herein and the understanding of the operations of the controllers provided by the present disclosure.

One of skill in the art, having the benefit of the disclosures herein, will recognize that the controllers, control systems and control methods disclosed herein are structured to perform operations that improve various technologies and provide improvements in various technological fields. Without limitation, example and non-limiting technology improvements include improvements in light operation, sanitization of the outer surface of the carrier, and sanitization of the carrier pocket of the customer unit.

Certain operations described herein include operations to interpret or determine one or more parameters. Interpreting and determining, as utilized herein, includes receiving values by any method known in the art, including at least receiving values from a datalink or network communication, receiving an electronic signal (e.g. a voltage, frequency, current, or PWM signal) indicative of the value, receiving a software parameter indicative of the value, reading the value from a memory location on a non-transient computer readable storage medium, receiving the value as a run-time parameter by any means known in the art, and/or by receiving a value by which the interpreted or determined parameter can be calculated, and/or by referencing a default value that is interpreted or determined to be the parameter value.

Certain systems are described following, and include examples of controller operations in various contexts of the present disclosure. The controller 600 is operable to sanitize the carrier 204 in a drive-through banking system. The controller 600 is operable to position the carrier 204 in either the customer station or the teller station of the drive-through banking system. The controller 600 is operable to close a door of either the customer station 202 or the teller station 302 that contains the carrier 204 therein. The controller 600 is operable to operate the light kit assembly 100 or 400 that contains one or more UV light sources 102 mounted on the mounting plate 104 in either the customer station 202 or the teller station 302 that contains the carrier 204, wherein the UV light from the one or more UV light sources 102 is directed towards the carrier 204 for a designated sanitization period.

Operation of the light kit assemblies 100 and 400 will now be described. For the sake of brevity, only the operation of light kit assembly 100 is described, however the operation of the light kit assembly 400 is substantially the same. The controller 600 controls the operation of the light kit assemblies 100 and 400. The light function or operation of the light kit assembly 100 can be enabled or disabled manually by a technician and/or automatically by the controller 600. When the light function is disabled, the drive-through banking system including the customer unit 202, teller unit 302, and the carrier 204 will operate without operation of the light kit assembly 100. In a disabled mode of operation, the light sources 102 are non-operational. Alternatively, in an enabled mode of operation, the light sources 102 are operational. To recall the carrier 204 to the customer unit 202, the carrier recall button on the customer unit 202 is engaged.

In a normal mode of operation, the controller 600 operates the light source 102 to turn OFF and the customer and/or bankers can use the drive-through banking system including the customer unit 202, teller unit 302, and the carrier 204. The indicator light 180, if present, is also turned OFF by the controller 600 to indicate that no sanitization is occurring.

The light kit assembly 100 is operable in either an automatic sanitization mode of operation or a manual sanitization mode of operation. The light kit assembly 100 being operable in an automatic sanitization mode of operation by the controller 600 will now be described. After a period of time of inactivity of the customer unit 202, the customer door of the customer unit 202 will automatically close. In one form, the period of time of inactivity is approximately three (3) minutes however in other forms the amount of time may be longer or shorter. Alternatively, after a designated number of uses of the customer unit 202 by customers, the customer door will automatically close and the automatic sanitization mode of operation will take place. Some examples of the designated number of uses are 1, 5, or 10 uses after which the automatic sanitization mode of operation takes place. Either before or after the customer door closes, the carrier 204 is recalled to the carrier pocket 200 of the customer unit 202. After the customer door closes and the carrier is located in the carrier pocket 200, the light sources 102 are turned ON for a designated sanitization period. The indicator light 180 is also turned ON for the designated sanitization period. In one form, the designated sanitization period is three (3) minutes however the designated sanitization period may be longer or shorter. After the designated sanitization period has passed, the light sources 102 and the indicator light 180, if present, will automatically turn off and the customer door will remain closed until engagement from a customer or a banker with the customer unit 202 or the teller unit 302. Beneficially, a short duration for the designated sanitization period is helpful to manage customers' expectations for quick service.

During the automatic sanitization mode of operation, the light function of the light sources 102 and the indicator light 180 can be interrupted by activity such as from a customer or banker wherein the light sources 102 and the indicator light 180 are turned OFF prior to having been ON for the duration of the designated sanitization period to operate in the interrupted mode of operation by the controller 600. Some examples of actions that will interrupt or stop the automatic sanitization mode of operation to stop the light source 102 and the indicator light 180 from emitting light include: opening and/or closing the teller door of the teller unit 302, pressing the customer send or call button, opening the customer door of the customer unit 202, recalling the carrier 204 to the customer unit 202, pressing the teller recall button, and/or recalling the carrier 204 to the teller unit 302. Activating the night or sanitizing lock switch 306 will also stop the light source 102 from emitting light and stop the automatic sanitization mode of operation. In one form, the carrier 204 is positioned in the carrier pocket 200 of the customer unit 202 during the automatic sanitization mode of operation when the automatic sanitization mode of operation is interrupted or stopped to recall the carrier 204 to the teller unit 302. In one form, activation of the night or sanitizing lock switch 306 also stops the light source 102 from emitting light, optionally recalls the carrier 204 to the teller unit 302, and optionally locks the light source 102 from further operation. Therefore, during the interrupted mode, the light function of the light source 102 does not operate for the entire duration of the designated sanitization period.

Alternatively, the light kit assembly 100 is operable in a manual sanitization mode of operation such that the customer or banker initiates the sanitization of the carrier 204. During the manual sanitization mode of operation, the night or sanitizing lock switch 306 is activated to turn ON. The controller 600 will then operably control the carrier 204 in the customer unit 202 or send the carrier 204 to the customer unit 202. The customer door may be open or closed. If the customer unit door is open, the controller 600 will operably close the customer door and the light source 102 and the indicator light 180 will turn ON for the designated sanitization period. In one form, the designated sanitization period is three (3) minutes however in other embodiments the designated sanitization period is longer or shorter. To interrupt or stop the light source 102 from emitting light for the entire designated sanitization period, the night or sanitizing lock switch 306 is turned OFF and the light source 102 and the indicator light 180 are turned OFF. The carrier 204 is then recalled to the teller unit 302 before the night or sanitizing lock switch 306 is turned ON to avoid the light source 102 and the indicator light 180 being turned ON. After the period of time of inactivity, the lane will go into normal night mode by sending the carrier 204 to the teller unit 302 and locking the light kit assembly 100.

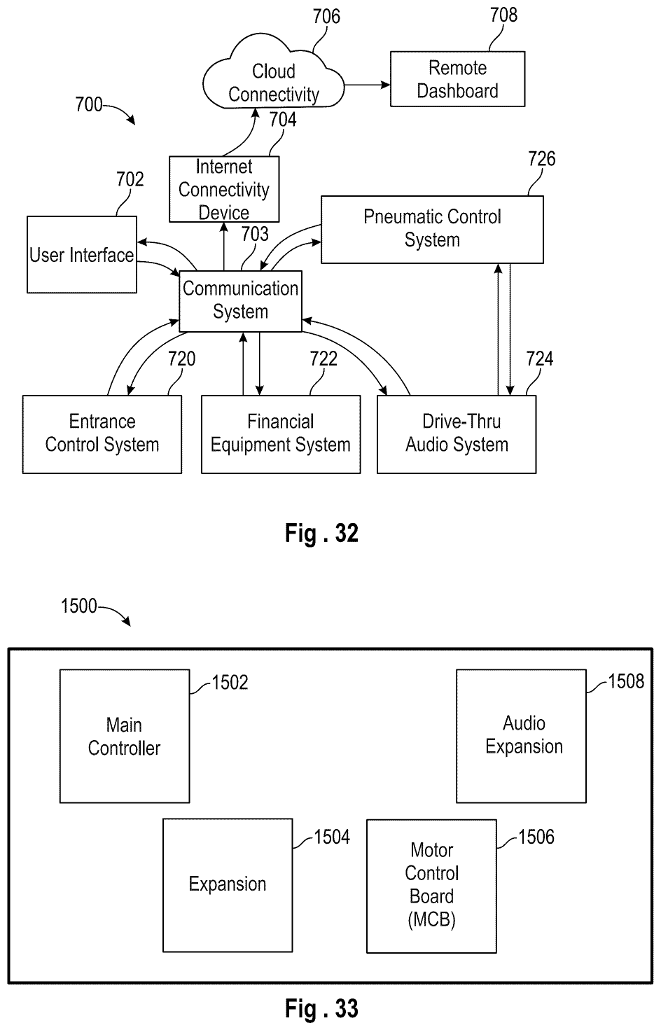

A control system 700 illustrated in FIG. 32 interacts with and receives device events from the controller 600. The control system 700 includes one or more of a user interface 702, a communication system 703, an internet connectivity device 704 and/or a cloud connectivity 706, an entrance control system 720, a financial equipment system 722, a drive-thru audio system 724, and a pneumatic control system 726.

The user interface 702 is configured to output or display various indications, messages, and/or prompts to an operator, which may be generated by the communication system 703. The user interface 702 is configured to provide various inputs to the communication system 703 based on various actions, which may include actions performed by an operator.

The communication system 703 can form a portion of a processing subsystem including one or more computing devices having memory, processing, and communication hardware. The communication system 703 may be a single device or a distributed device, and the functions of the communication system 703 may be performed by hardware or by instructions encoded on computer readable medium. The communication system 703 is in communication with the user interface 702, the internet connectivity device 704, the cloud connectivity 706, the remote dashboard 708, the entrance control system 720, the financial equipment system 722, the drive-thru audio system 724, and the pneumatic control system 726. The communication system 703 can communicate through direct communication, communication over a datalink, and/or through communication with other controllers or portions of the processing subsystem that provide sensor and/or actuator information such as with the controller 600.

In one embodiment, the control system 700 includes the internet connectivity device 704 for internet connectivity, such as wireless, cable, fiber, DSL, and/or dial-up, however other types of internet connectivity may be suitable for the communication system 703 to a remote dashboard 708. In one embodiment, the communication system 703 is connected to the remote dashboard 708 via the cloud connectivity 706. The cloud connectivity 706 can include a network of fiber and 5G to connect tools, applications, machines, and other technologies to cloud service providers (CSPs) or other third parties that provide cloud resources like processing or computing, storage, platforms, and application hosting. The remote dashboard 708 is configured to output or display various indications, messages, and/or prompts to an operator, which may be generated by or sent to the communication system 703 from any of the user interface 702, the entrance control system 720, the financial equipment system 722, the drive-thru audio system 724, and the pneumatic control system 726.

A plurality of universal boards 1500 are illustrated in FIG. 33 wherein each of the universal boards 1500 may be specific for assembly with a particular piece of equipment in the drive up banking system. For example, the plurality of universal boards 1500 can include a main controller universal board 1502 that can be assembled with the controller 600 or the communications system 703, an expansion universal board 1504 that can be assembled with the entrance control system 720 which can include the customer station 202 and the teller station 302, a motor control board 1506 that can be assembled with the pneumatic control system 726, and an audio expansion board 1508 that can be assembled with the drive-thru audio system 724. The plurality of universal boards 1500 are configured to communicate to one or more back end services. In an alternative embodiment, the plurality of universal boards 1500 can include a main controller universal board 2502 that can be assembled with the controller 600 or the communications system 703 and the entrance control system 720. In this embodiment, the expansion universal board is not required.

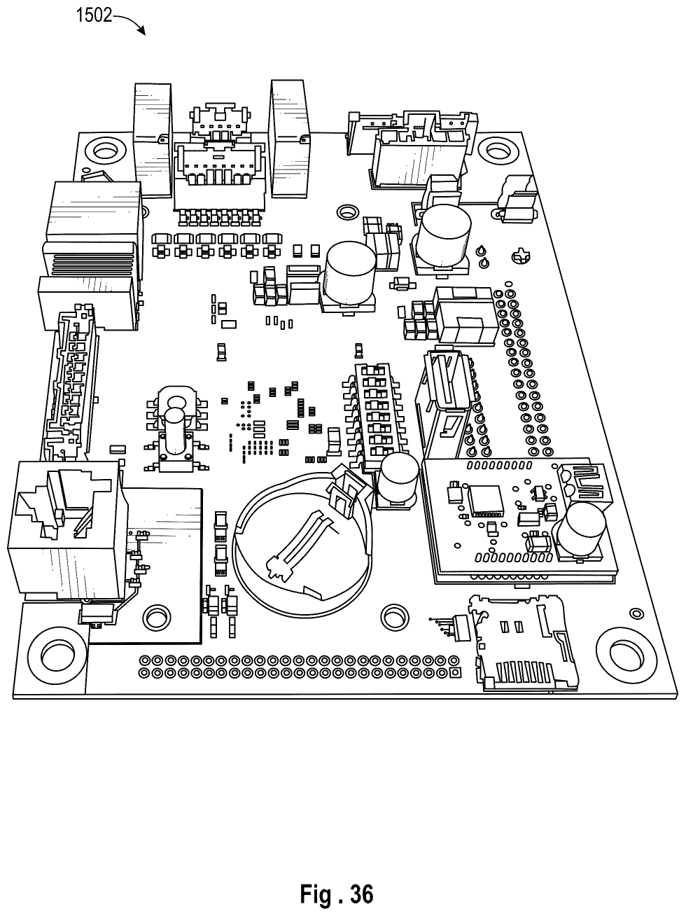

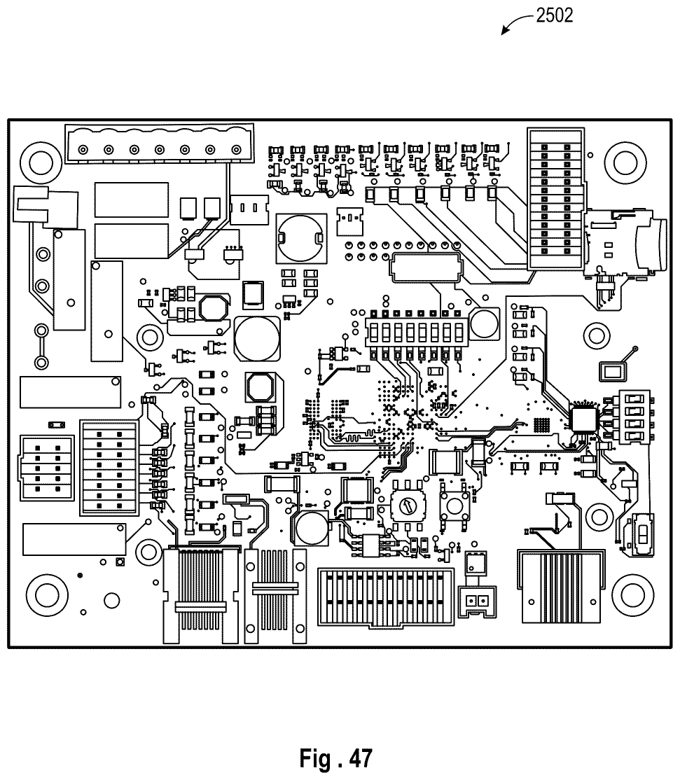

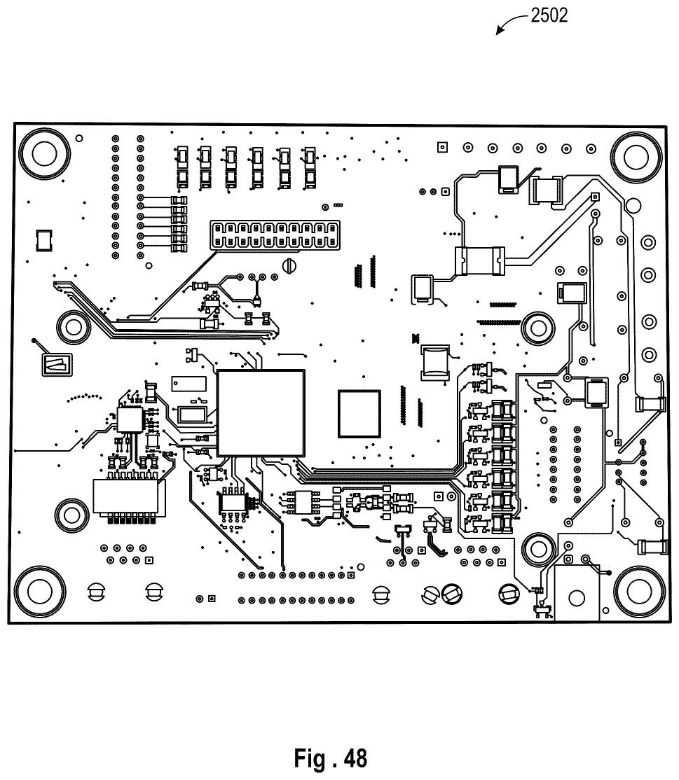

One exemplary embodiment of the main controller universal board 1502 is illustrated in FIG. 36. In other embodiments, the main controller universal board 1502 is configured differently such as described in FIGS. 47 and 48. The main controller universal board 1502 includes a processor and main controller and one or more of a USB port, an expansion USB port, a micro-SD card, a CAN bus, an Ethernet connection, one or more configuration switches, one or more pushbuttons, one or more door motor drivers, input/output, one or more serial ports, and a LCD display port. The main controller universal board 1502 can be operably connected to the expansion board 1504 and/or to the motor control board 1506. The main controller universal board 1502 is configured to communicate messages and/or information to the communication system 703.

One exemplary embodiment of the main controller universal board 2502 is illustrated in FIGS. 47 and 48. The main controller universal board 2502 includes a processor and main controller and one or more of a USB port, an expansion USB port, a micro-SD card, a CAN bus, an Ethernet connection, one or more configuration switches, one or more pushbuttons, one or more door motor drivers, input/output, one or more serial ports, and a LCD display port. The main controller universal board 2502 can be operably connected to the entrance control system 720. The main controller universal board 2502 can be operably connected to the motor control board 1506. The main controller universal board 2502 is configured to communicate messages and/or information to the communication system 703 and the entrance control system 720.

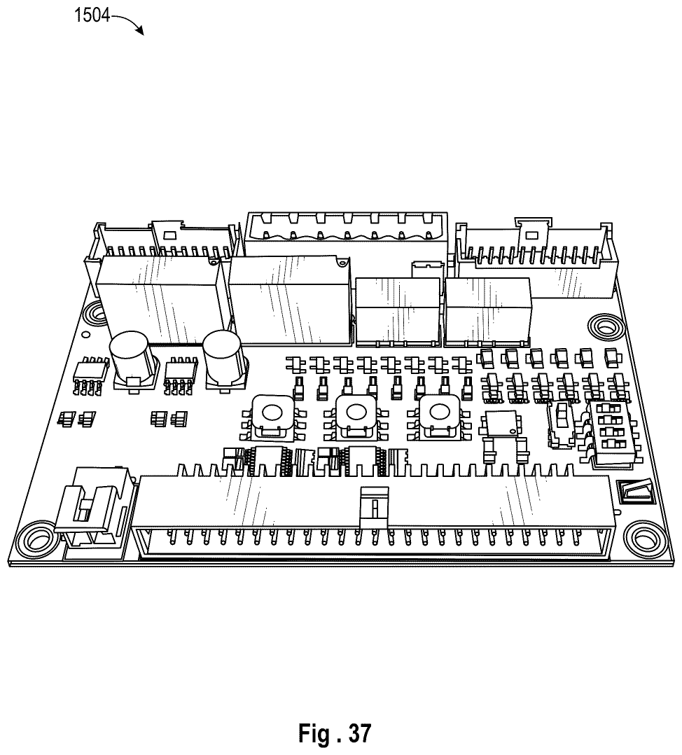

One exemplary embodiment of the expansion universal board 1504 is illustrated in FIG. 37. In other embodiments, the expansion universal board 1504 is configured differently. The expansion universal board 1504 connects to the main controller universal board 1502 via an expansion ribbon cable or by other means known in the industry. The expansion universal board 1504 is assembled with the customer station 202 and/or the teller station 302 of the entrance control system 720. The expansion universal board 1504 can operate as the controller 600.

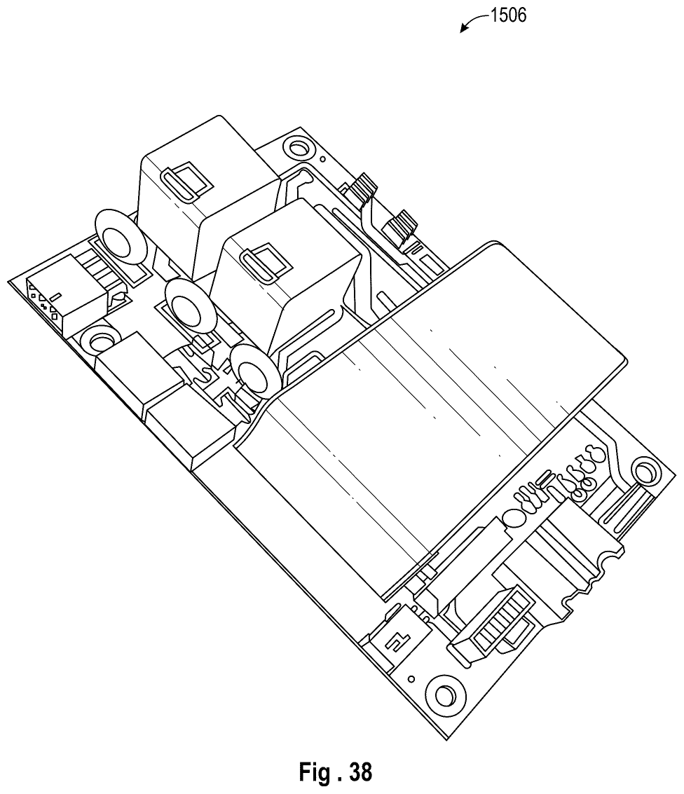

One exemplary embodiment of the motor control board 1506 is illustrated in FIG. 38. In other embodiments, the motor control board 1506 is configured differently. The motor control board 1506 is connected to the main controller universal board 1502 or 2502 via a CAN bus or by other means known in the industry. The motor control board 1506 can supply low-voltage such as 24VDC to the main controller universal board 1502 or 2502. The motor control board 1506 controls other pressure and/or vacuum motors. The motor control board 1506 is configured to mount directly on a blower or other piece of equipment of the entrance control system 720.

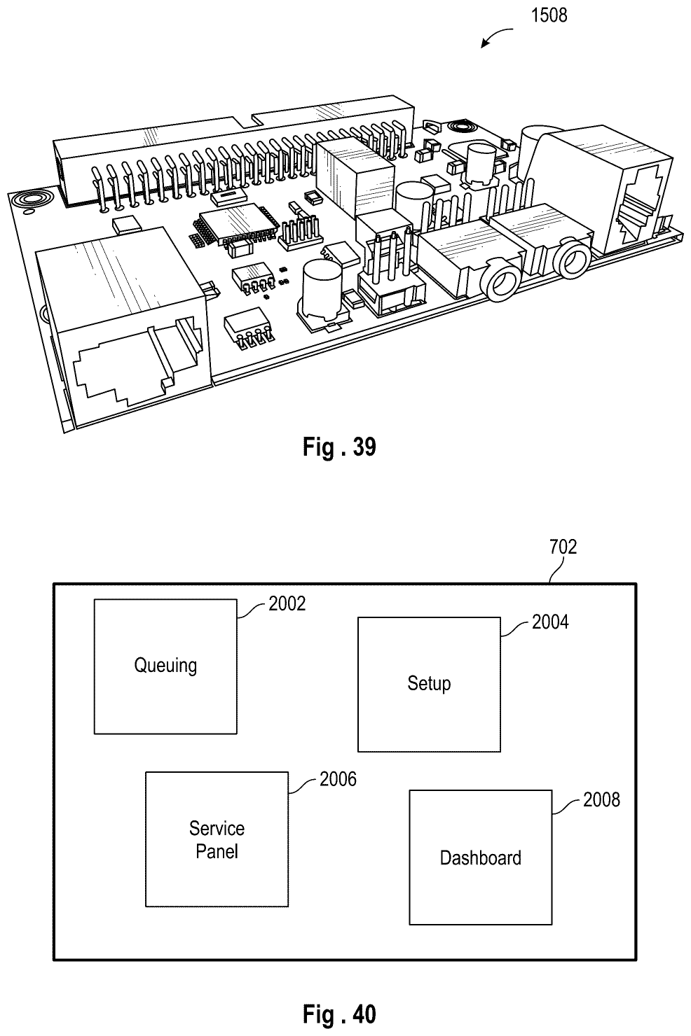

One exemplary embodiment of the audio expansion universal board 1508 is illustrated in FIG. 39. In other embodiments, the audio expansion universal board 1508 is configured differently. The audio expansion universal board 1508 is assembled with the drive-thru audio system 724.

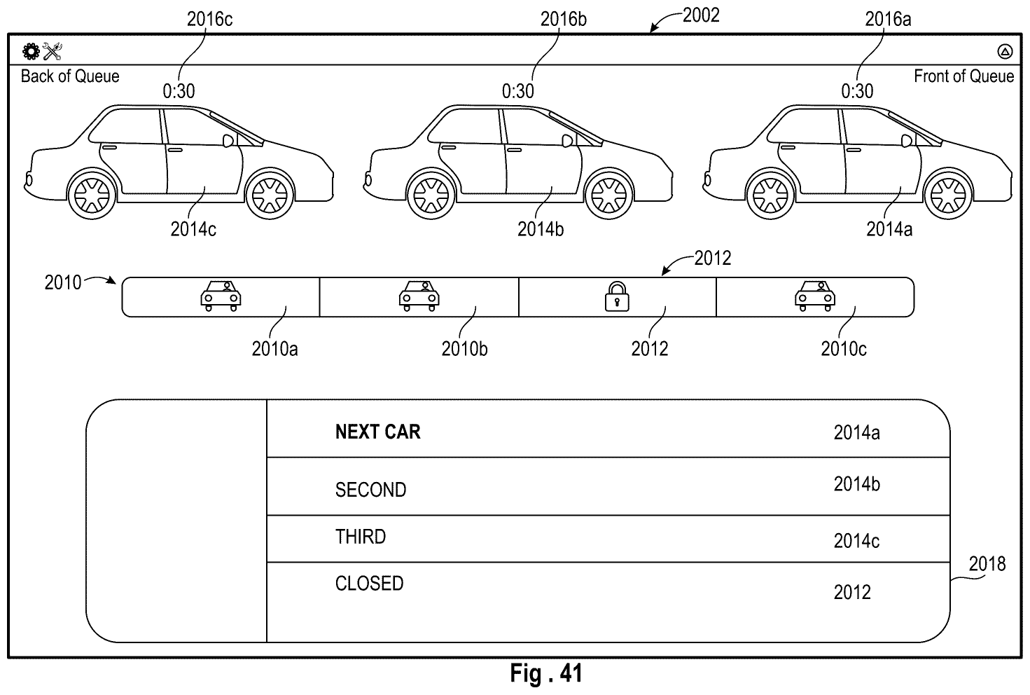

The plurality of universal boards 1500 support the integration of the “universal board enabled” equipment or systems into the control system 700 for diagnostics, management, service, technical support, analytics, and preventative maintenance of the universal board enabled equipment of the drive up banking system. The universal board enabled equipment communicates to the communication system 703 that can be located on the controller 600 or another controller via an IoT communications protocol with messages in a format suitable for each of universal board enabled equipment. These messages can be consolidated and displayed on the user interface 702 and/or the remote dashboard 708 in a format suitable for each of the universal board enabled equipment. For example, drive-up banking equipment can display a drive-thru equipment identifier, a drive-thru lane priority queueing in order to service customers in the correct order such as based on when the customer initiated a transaction or some other desired order, analytics of a financial institution's choice such as a number of daily transactions or an average customer wait time on the user interface 702 and/or the remote dashboard 708 as further discussed in FIGS. 41-43. In some embodiments, a locally available “service panel” such as the user interface 702 and/or the remote dashboard 708 can be used to view all the messages communicated to the communication system 703. An operator can also communicate messages to the communication system 703 via the user interface 702 and/or remote dashboard 708.

The entrance control system 720 is configured to determine and/or display a lane status of the drive-up lane for customers to access a drive up banking system such as described above. The entrance control system 720 communicates with the communication system 703 to send the lane status to the communication system 703. The communication system 703 communicates with the entrance control system 720 to change and/or display the lane status. The lane status determined by the entrance control system 720 can include any of financial messages such as open/closed, automated teller machine (ATM), commercial, and/or personal teller that indicate or describe the intended use for the physical drive-up lane. Other messages than the lane status can also be displayed. The lane status being open/closed indicates whether any of the bank lanes are open allowing customers to select a lane to drive through to conduct their business or closed to indicate that customers should not select that particular lane to conduct any business. As discussed previously, normal operation mode indicates that the carrier 204 is operable to move between the customer station 202 and the teller station 302 and the carrier 204 is accessible for use by customers or bankers. In the normal operation mode, the lane status is open. When sanitization of the carrier 204 is desired, the carrier 204 is returned or positioned in the carrier pocket of the customer unit 202 and the drive-up lane is activated to the night or sanitizing mode of operation. In the night or sanitizing mode of operation, the lane status is closed. The lane status being automated teller machine (ATM) indicates that the ATM is associated with that particular bank lane. The lane status being commercial or personal indicates that the type of banking accessible in that particular bank lane is either commercial such as for a business or personal such as for an individual.

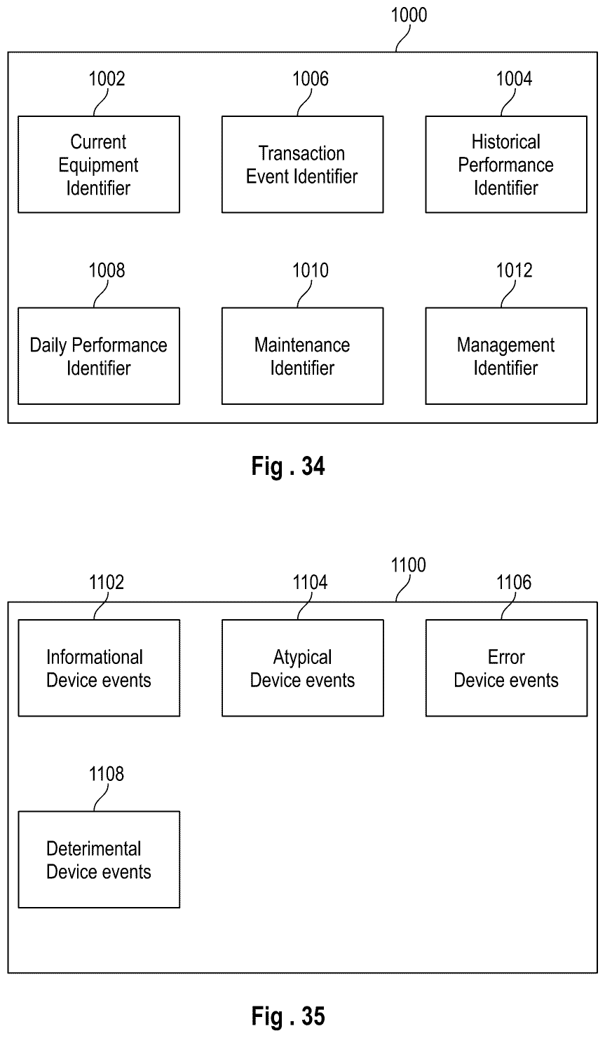

The financial equipment system 722 is configured to determine a plurality of equipment identifiers 1000 such as illustrated in FIG. 34 for a particular piece of equipment that includes one of the expansion universal board 1504. For example, the plurality of equipment identifiers 1000 can include a current equipment identifier 1002, a historical performance identifier 1004, a transaction event identifier 1006, a daily performance identifier 1008, a maintenance identifier 1010, and a management identifier 1012. The plurality of equipment identifiers 1000 can include additional equipment identifiers as desired. The financial equipment system 722 communicates the plurality of equipment identifiers 1000 to the communication system 703.

The current equipment identifier 1002 is the latest or real-time status of a particular piece of equipment that includes the expansion universal board 1504. The historical performance identifier 1004 relates to a historical analysis of a life-expectancy or end-of-life based on a maximum amount of expected and/or anticipated wear of the particular piece of equipment that includes the expansion universal board 1504 before breakage or malfunction of the particular piece of equipment. For example, switches have a limited number of on/off cycles or motors have a limited run-time life before breakage or malfunction and the historical performance identifier 1004 includes these metrics. The universal board-enabled equipment of the entrance control system 720, the financial equipment system 722, the drive-thru audio system 724, the pneumatic control system 726, and/or the customer and teller units 202 and 302, respectively, may consist of various components, such as switches, motors, doors, turbines, that each have a limited life expectancy that corresponds to the historical performance identifier 1004. Each piece of equipment that includes the expansion universal board 1504 has its own key metrics that are monitored as the daily performance identifier 1008. Each piece of equipment that includes the expansion universal board 1504 has its own scheduled maintenance that can be performed as the maintenance identifier 1010. Each piece of equipment that includes the expansion universal board 1504 has transactions that are performed and recorded as the transaction event identifier 1006. Each piece of equipment that includes the expansion universal board 1504 can be controlled or managed by a user and these actions are logged by the management identifier 1012.

The drive-thru audio system 724 is configured to enable an audio message of sound to be recorded, transmitted, or reproduced to the particular piece of equipment that includes the audio expansion universal board 1508. The drive-thru audio system 724 communicates with the communication system 703 to send the audio message to the communication system 703. The communication system 703 communicates with the drive-thru audio system 724 to change and/or emit the audio message. The drive-thru audio system 724 is configured to output or display any sounds, alerts, messages, or alarms which may be generated by the communication system 703 based on the entrance control system 720, the financial equipment system 722, and the pneumatic control system 726. In one embodiment, the drive-thru audio system 724 includes a speaker and a user interface for generating and/or displaying.

The pneumatic control system 726 includes the motor control board 1506 that can be assembled with a pneumatic point-to-point transport system that includes a tube to connect the teller unit 302 and the customer unit 202. In one embodiment, the pneumatic point-to-point transport system includes a blower and/or a motor to carry or transport the carrier 204 between the teller and customer units 302 and 202, respectively. The pneumatic point-to-point transport system can also include one or more video cameras, two-way video system, and/or microphone. The pneumatic control system 726 determines one or more pneumatic control steps are performed in the motor control board 1506. The pneumatic control system 726 is configured to communicate with the drive-thru audio system 724. However, it is not necessary for the pneumatic control system 726 to receive user input or operator instructions to operate. The pneumatic control system 726 communicates with the communication system 703 to send a pneumatic status of the pneumatic point-to-point transport system to the communication system 703. The communication system 703 communicates with the pneumatic control system 726 to modify operation of the pneumatic point-to-point transport system.

The communication system 703 receives a plurality of device events 1100 from any of the entrance control system 720, the financial equipment system 722, the drive-thru audio system 724, and/or the pneumatic control system 726. The plurality of device events 1100 include one or more of a plurality of informational device events 1102, a plurality of atypical device events 1104, a plurality of error device events 1106, and/or a plurality of detrimental device events 1108 as illustrated in FIG. 35. The communication system 703 determines based on the plurality of device events 1100 one or more messages that indicate a system status to display on the user interface 702 and/or the remote dashboard 708 as described in more detail below.

The plurality of informational device events 1102 are descriptive of a current state of any of the entrance control system 720, the financial equipment system 722, the drive-thru audio system 724, the pneumatic control system 726, and/or including the customer and teller units 202 and 302, respectively. The plurality of informational device events 1102 are not associated with warnings or errors for any of the universal board-enabled equipment. Additionally, the plurality of informational device events 1102 enable the universal board-enabled equipment to continue operation. The plurality of informational device events 1102 can be displayed on the user interface 702 and/or the remote dashboard 708.

The plurality of informational device events 1102 include any of the following events for the customer and teller units 202 and 302, respectively: customer send button was pressed, customer send button was released, customer call button was pressed, customer call button was released, customer mic was muted, customer mic was unmuted, all available pressure motors were turned on, all available pressure motors were turned off, all available vacuum motors were turned on, all available vacuum motors were turned off, customer carrier was detected, customer carrier was no longer detected, customer door button was turned on, customer door button was turned off, customer door open switch was detected, customer door open switch is no longer detected, customer door closed switch was detected, customer door closed switch is no longer detected, customer video was turned on, customer video was turned off, customer safety bar was detected, customer safety bar is no longer detected, lane open was turned on, lane open was turned off, customer door was auto closed, manual teller door was closed to send, manual teller door was opened, manual teller recall button was pressed, manual teller recall button was released, manual teller night lock was turned on, manual teller night lock was turned off, manual teller carrier was detected, manual teller carrier was no longer detected, motorized teller send button was pressed, motorized teller send button was released, motorized teller recall button was pressed, motorized teller recall button was released, motorized teller night lock was turned on, motorized teller night lock was turned off, motorized teller carrier was detected, motorized teller carrier was no longer detected, motorized teller door motor was turned on, motorized teller door motor was turned off, motorized teller door open switch was detected, motorized teller door open switch is no longer detected, motorized teller door closed switch was detected, motorized teller door closed switch is no longer detected, motorized teller safety bar was detected, motorized teller safety bar is no longer detected, a vehicle was detected, and/or a vehicle has left.

The plurality of informational device events 1102 can also include any of the following events for the customer and teller units 202 and 302, respectively: customer unit preventative maintenance (PM) was performed, teller unit preventative maintenance (PM) was performed, the lane status is acceptable, the lane status has one or more warnings but no errors, a UV Light relay output has turned on, UV Light relay output has turned off, airflow turned on, airflow turned off, heater relay output has turned on, heater relay output has turned off, an event indicating the universal board-enabled equipment has powered up, event indicating the universal board-enabled equipment is performing a controlled and desired shutdown, event to communicate a “user message” to the service panel telling the user to perform some action such as closing the teller door, an event indicating that a workflow was halted, and/or an event to indicate that a device was detected or removed.

The plurality of atypical device events 1104 includes warnings that indicate an event has occurred that is abnormal or unusual but the event does not affect functionality of the universal board-enabled equipment of the entrance control system 720, the financial equipment system teller units 202 and 302, respectively. As such, the universal board-enabled equipment continues operation even though the atypical event 1104 has been detected.

The plurality of atypical device events 1104 include any of the following as measured against or relative to a past or most recent number of flights of the carrier: the time required for the carrier to go out to the customer unit was higher than the average, the time it took for the carrier to come into the teller was higher than the average, the time it took for the customer door to close was higher than the average, the time it took for the customer door to open was higher than the average, and/or the time it took for the motorized teller door to open was higher than the average. In one embodiment, the time is between 25% and 50% higher than any of the previous averages. In one embodiment, the number of past or most recent flights of the carrier 204 is 50 or more.

The plurality of atypical device events 1104 include any of the following: the customer door closed switch was not detected, the carrier was not detected at the teller side and it was expected, the carrier was not detected at the customer side and it was expected, the motorized teller safety bar was detected, the teller door was opened while the carrier was on its way to the customer side, the motor was turned off before arrival, early pressure was turned on but the carrier in workflow failed while the customer door was closing, the customer door open switch was expected to be made but it was not detected, the customer door closed switch was expected to be made but it was not detected. For some embodiments, the number of detections of events is greater than 10 times before triggering the status of the atypical device event 1104.

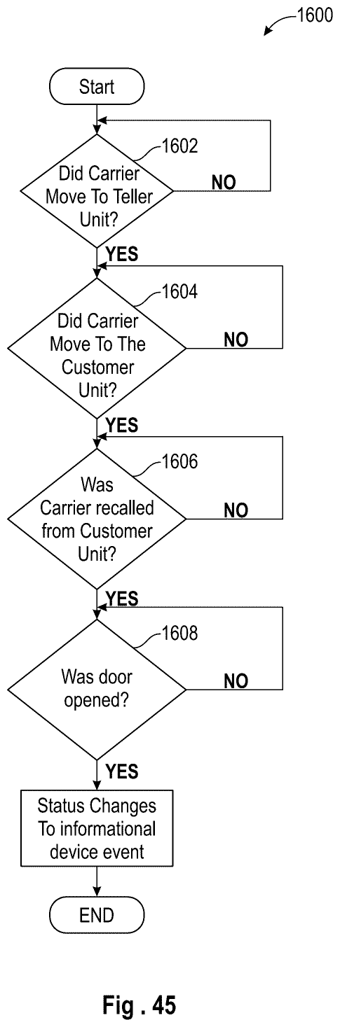

The plurality of atypical device events 1104 will not be cleared from the system status until a clearing workflow 1600 as illustrated in FIG. 45 occurs without incident. The clearing workflow 1600 includes operation of the carrier 204 between the customer and teller units 202 and 302, respectively, including a customer send step 1602 at the customer unit 202 to send the carrier 204 to the teller unit 302, a teller send step 1604 to send the carrier 204 to the customer unit 202, a teller recall step 1606 to recall the carrier 204 from the customer unit 202, and an auto-door open step 1608 to open a door of either the customer or the teller units 202 and 302, respectively. If the carrier 204 does not successfully move according to any of steps 1602, 1604, 1606, or 1608, then the atypical device event 1104 remains as the system status. If the carrier 204 does successfully move according to all of the steps 1602, 1604, 1606, or 1608, then the atypical device event 1104 is cleared from the system status such that the system status returns to any of the informational device events 1102.

The plurality of error device events 1106 are errors that indicate the communication system 703 has detected or received an error or malfunction in the universal board-enabled equipment but the error or malfunction will not affect functionality of the universal board-enabled equipment of the entrance control system 720, the financial equipment system 722, the drive-thru audio system 724, the pneumatic control system 726, and/or the customer and teller units 202 and 302, respectively. As such, the universal board-enabled equipment continues operation even though the error device event 1106 was detected.

The plurality of error device events 1106 include any of the following events: the customer unit universal board failed to communicate with the motorized teller board, the customer unit universal board failed to communicate with the first one of motor control board 1506, the customer unit universal board failed to communicate with the second one of the motor control board 1506, the customer unit universal board failed to communicate with the customer unit input/output expander board, the motorized teller unit had an input/output failure, the first one of the motor control board 1506 had an input/output failure, the second one of the motor control board 1506 had an input/output failure, the customer unit universal board had an input/output failure, the customer unit call button is detected as being stuck, the customer unit send button is detected as being stuck, the teller unit send button is detected as being stuck, the teller unit recall button is detected as being stuck, the manual teller door closed switch (send button) is detected as being stuck, the manual teller door open switch (send button) is detected as being stuck, the motorized teller door was attempted to be closed to start a workflow but the door never got to the closed position, the requested workflow failed to run because a required device failed, the carrier was expected to move and it ended up in the same position it started, the customer unit universal board failed to communicate with the third one of the motor control board 1506, the customer unit universal board failed to communicate with the fourth one of the motor control board 1506, the third one of the motor control board 1506 had an input/output failure, the fourth one of the motor control board 1506 had an input/output failure.

The plurality of error device events 1106 can also include the customer safety bar or the motorized teller safety bar was detected and ended the requested workflow which occurs when the safety bar fails during two or more consecutive workflow attempts. The first time the safety bar fails it will not trigger anything and the second time the safety bar fails it will be an error device event 1106. Moreover, the plurality of error device events 1106 will not be cleared from the system status until the clearing workflow 1600 happens without incident. The clearing workflow 1600 includes operation of the carrier 204 between the customer and teller units 202 and 302, respectively, including the customer send step 1602 at the customer unit 202 to send the carrier 204 to the teller unit 302, the teller send step 1604 to send the carrier 204 to the customer unit 202, the teller recall step 1606 to recall the carrier 204 from the customer unit 202, and the auto-door open step 1608 to open a door of either the customer or the teller units 202 and 302, respectively. If the carrier 204 does not move according to any of steps 1602, 1604, 1606, or 1608, then the error device event 1106 remains as the system status. If the carrier 204 does move according to all of steps 1602, 1604, 1606, or 1608, then the error device event 1106 changes to the informational device event.

The plurality of detrimental device events 1108 are events and errors that indicate a failure in the universal board-enabled equipment of the entrance control system 720, the financial equipment system 722, the drive-thru audio system 724, the pneumatic control system 726, and/or the customer and teller units 202 and 302, respectively, has occurred. The plurality of detrimental device events 1108 include any of a disconnected circuit board, a failed circuit board, a hardware failure, the general purpose input/output pin failed to initialize, a delay is initiated in the general purpose input/output command could not be implemented, the general purpose input/output command was initiated on an invalid input/output, a local general purpose input/output was commanded to change state and failed, a delay is initiated and the general purpose input/output command could not be implemented, a universal board undefined error, an input on the universal board is attempting to be read but the universal board is not initialized, the universal board is not detected, a universal general purpose input/output command was initiated on an invalid input/output id or expander port, a queued output on the universal board was attempted but the queue was full, failure to read from the input/output expander on the universal board, failure to write to the input/output expander on the universal board, a general CAN Bus Error Occurred, a command was attempted on a CAN device that is not currently supported or the CAN module has not been initialized, a reply was expected from a CAN connected device but was never received, a command was attempted on the motor control board 1506 but the motor control board 1506 is undetected on the CAN bus, or failure to send a CAN bus command to the motor control board 1506.

Turning now to FIG. 40 is an exemplary embodiment of the user interface 702 that includes a queuing screen 2002, a setup screen 2004, a service panel 2006, and the remote dashboard 708. Each of the queuing screen 2002, the setup screen 2004, and the service panel 2006 is described in more detail below.

Turning now to FIG. 41 is an exemplary embodiment of the queuing screen 2002 that tracks and displays a current queue 2010 on the user interface 702. The current queue 2010 includes a number of queues 2010a, 2010b, and 2010c that correspond to a plurality of lanes 2014a, 2014b, and 2014c, respectively, at the drive-through banking system. The plurality of lanes 2014a, 2014b, and 2014c include the specific lane that a vehicle can enter for the drive-up banking system. The current queue 2010 can also include a blocked or locked lane 2012 that indicates that the drive-through banking system is not operable for that particular lane of traffic. The queuing screen 2002 includes a plurality of estimated times 2016a, 2016b, and 2016c that correspond to the amount of time required to service a vehicle that enters the lanes 2014a, 2014b, and 2014c, respectively. The plurality of estimated times 2016a, 2016b, and 2016c can be adjusted as desired. In some embodiments, the queuing screen 2002 includes an ordered summary 2018 to track the order of vehicles in the plurality of lanes 2014a, 2014b, and 2014 that are serviced. In other embodiments, the bank's performance as measured relative to the plurality of estimated times 2016a, 2016b, and 2016c can also be tracked and displayed.

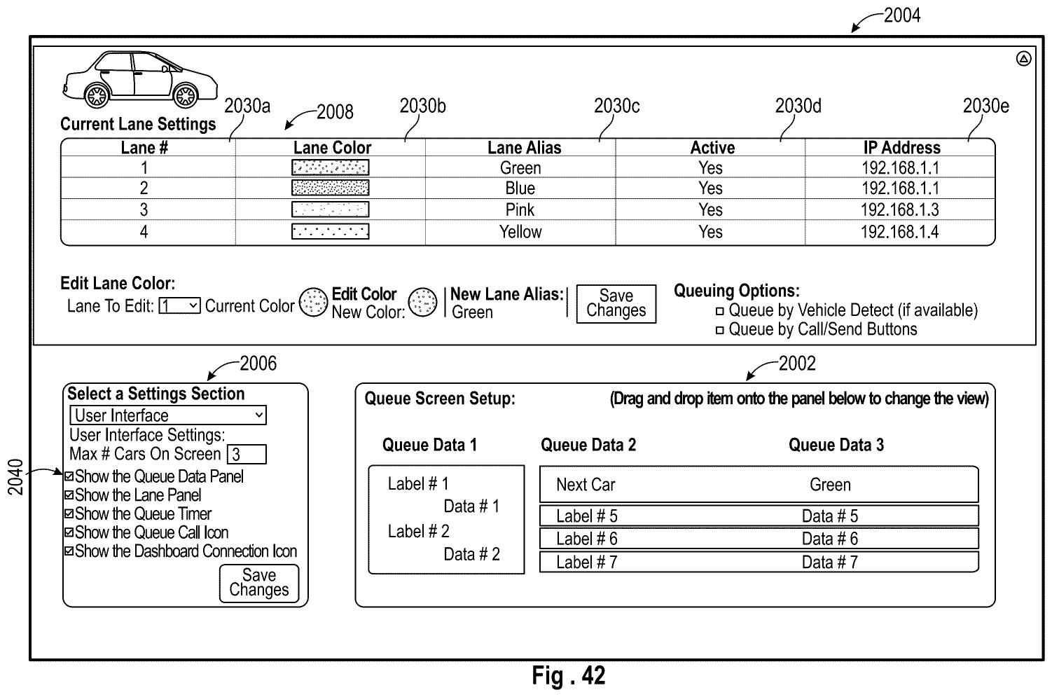

Turning now to FIG. 42 is an exemplary embodiment of the setup screen 2004 that includes a default setting of each of the queuing screen 2002, the remote dashboard 708, and the service panel 2006. Each of the queuing screen 2002, the user interface 702, the remote dashboard 708, and the service panel 2006 can be configured as desired by the user. The default setting allows for minimal setup during installation of the control system 700. For example, the user interface 702 can include a plurality of individual lane settings 2030a-2030e that include any of a lane number 2030a, a lane color 2030b, a lane alias 2030c, a status of operability of the lane 2030d, and an IP address 2030e, to name a few examples of individual lane settings 2030a-2030e. The user interface 702, the remote dashboard 708, and the queuing screen 2002 are each configurable. In one form, the user uses a drag and drop technique to include any of a plurality of settings 2040 from the service panel 2006. Some exemplary settings 2006 include a location that the settings will be displayed such as on the user interface 702, a service incident that indicates a service technician is needed (not illustrated), and the remote dashboard 708, to name a few.

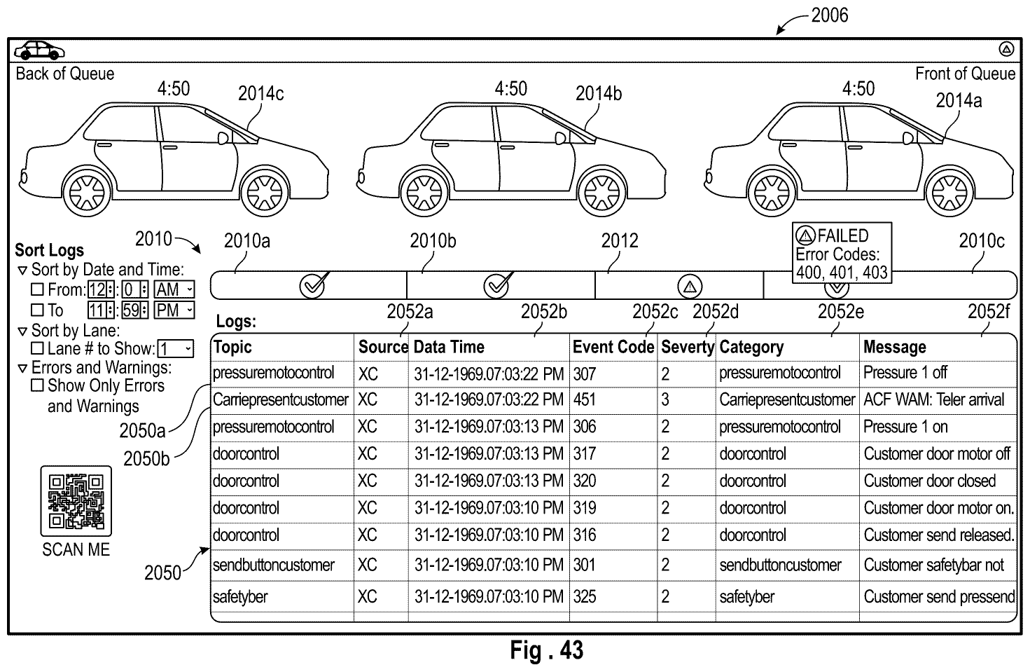

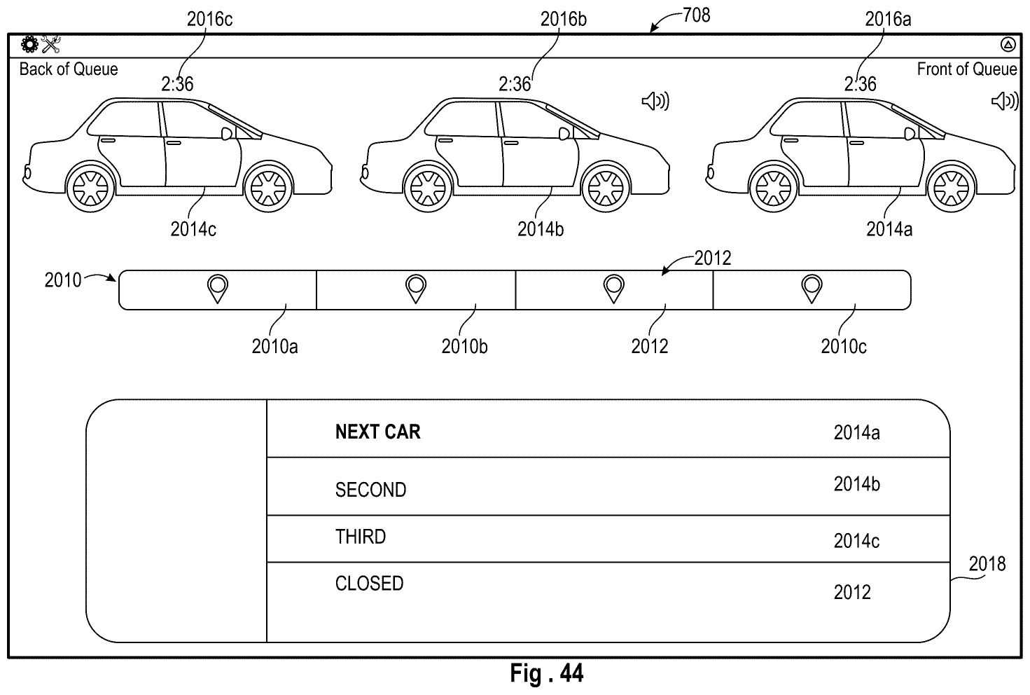

Turning now to FIG. 43 is an exemplary embodiment of the service panel 2006 that can be displayed on the user interface 702 and/or the remote dashboard 708. The service panel 2006 includes a plurality of logs 2050 wherein each of the logs 2050 corresponds to a particular piece or part of equipment associated with the drive-through banking system. For example, the plurality of logs 2050 includes a pressure motor control log 2050a. The pressure motor control log 2050a includes a source 2052a of the pressure motor, a timestamp of current status 2052b of the pressure motor, an event code 2052c that indicates an issue or malfunction with the pressure motor, a severity rating 2052d of the issue or malfunction, a category 2052e of the location of the pressure motor in the drive-through banking system, and a message 2052f that corresponds to the event code. A service technician or other user is able to review the plurality of logs 2050. The plurality of logs 2050 are able to be sorted by any of the source 2052a, the timestamp of current status 2052b, the event code 2052c, the severity rating 2052d, the category 2052e, and the message 2052f.

In some embodiments, the service panel 2006 displays the current queue 2010 that includes the plurality of queues 2010a, 2010b, and 2010c that correspond to the plurality of lanes 2014a, 2014b, and 2014c, respectively, and the blocked or locked lane 2012. The bank operators or tellers are able to continue servicing vehicles in the plurality of lanes 2014a, 2014b, and 2014c by the current queue 2010 while the service technician views the plurality of logs 2050.

Turning now to FIG. 44 is an exemplary embodiment of the remote dashboard 708 that can display any of the user interface 702, the queuing screen 2002, the setup screen 2004, and the service panel 2006. In the illustrated embodiment, the remote dashboard 708 displays the queuing screen 2002.

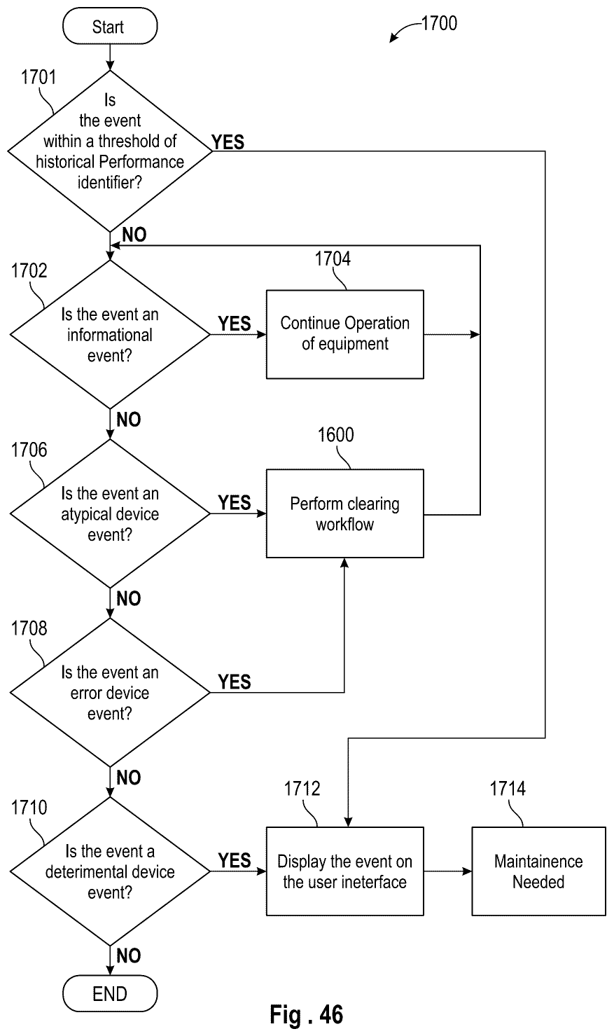

Turning now to FIG. 46 is one embodiment of a preventative maintenance module 1700 operable with the control system 700. The preventative maintenance module 1700 begins with step 1701 to determine if the current equipment identifier 1002 or event logged by the universal board-enabled equipment of the entrance control system 720, the financial equipment system teller units 202 and 302, respectively, is within a performance threshold of the historical performance identifier 1004. The performance threshold is a boundary or limit that is near but less than the historical performance identifier 1004. In one embodiment, the performance threshold is time based to correspond with the historical performance identifier 1004 that is the life-expectancy or end-of-life based on the maximum amount of time that the particular piece of equipment is determined to function before repair is needed. In another embodiment, the performance threshold is cycle based to correspond to the historical performance identifier 1004 that is a maximum number of on/off cycles or maximum number of operations before breakage or malfunction. The performance threshold is dependent on the particular piece of equipment and can also be modified by a user. If the event is within the performance threshold of the historical performance identifier 1004, then the preventative maintenance module 1700 continues to step 1712 as described below. Step 1712 includes displaying the event on the user interface 702. The preventative maintenance module 1700 determines when components need to be replaced in a more intelligent fashion, decreasing the downtime of universal board-enabled equipment of the entrance control system 720, the financial equipment system 722, the drive-thru audio system 724, the pneumatic control system 726, and/or the customer and teller units 202 and 302, respectively, and increasing the useful life of these products. Communicating via the user interface 702 only at the appropriate times, i.e., when the current equipment identifier 1002 is within the performance threshold reduces the number of required trips to the site for non-essential maintenance and reduces the overall cost of ownership of the equipment.

If the event is not within the performance threshold of the historical performance identifier 1004, then the preventative maintenance module 1700 continues to step 1702. At step 1702, the preventative maintenance module 1700 determines if the current equipment identifier 1002 or event logged by the universal board-enabled equipment of the entrance control system 720, the financial equipment system 722, the drive-thru audio system 724, the pneumatic control system 726, and/or the customer and teller units 202 and 302, respectively, is any of the informational device events 1102. If the event is the informational device event 1102, then the preventative maintenance module 1700 continues to step 1704 wherein the universal board-enabled equipment of the entrance control system 720, the financial equipment system 722, the drive-thru audio system 724, the pneumatic control system 726, and/or the customer and teller units 202 and 302, respectively, continue operation.

If the event is not the informational device event 1102, then the preventative maintenance module 1700 continues to step 1706 to determine if the current equipment identifier 1002 or event logged by the universal board-enabled equipment of the entrance control system 720, the financial equipment system 722, the drive-thru audio system 724, the pneumatic control system 726, and/or the customer and teller units 202 and 302, respectively, is any of the atypical device events 1104. If the current equipment identifier 1002 or event is the atypical device event 1104, then the preventative maintenance module 1700 continues to step 1600 wherein the clearing workflow 1600 is performed.

If the current equipment identifier 1002 or event is not the atypical device event 1104, then the preventative maintenance module 1700 continues to step 1708 to determine if the event logged by the universal board-enabled equipment of the entrance control system 720, the financial equipment system 722, the drive-thru audio system 724, the pneumatic control system 726, and/or the customer and teller units 202 and 302, respectively, is any of the error device events 1106. If the event is the error device event 1106, then the preventative maintenance module 1700 continues to step 1600 wherein the clearing workflow 1600 is performed.

If the event is not the error device event 1102, then the preventative maintenance module 1700 continues to step 1710 to determine if the event logged by the universal board-enabled equipment of the entrance control system 720, the financial equipment system 722, the drive-thru audio system 724, the pneumatic control system 726, and/or the customer and teller units 202 and 302, respectively, is any of the detrimental device events 1108.

If the event is the detrimental device event 1108, then the preventative maintenance module 1700 continues to step 1712 wherein either the detrimental device event 1108 or the event from step 1701 is displayed on the user interface 702 and/or the remote dashboard 708. The plurality of detrimental device events 1108 are events and errors that indicate a failure in the universal board-enabled equipment of the entrance control system 720, the financial equipment system 722, the drive-thru audio system 724, the pneumatic control system 726, and/or the customer and teller units 202 and 302, respectively, has occurred. If the event is the detrimental device event 1108 or the event from step 1701, then the preventative maintenance module 1700 continues to step 1714 to determine the type of specific maintenance needed on the failed equipment. Step 1714 may include discontinuation of operation of the failed equipment.

If the event is not the detrimental device event 1108 or the event from step 1701, then the preventative maintenance module 1700 ends or in some embodiments, the event is displayed on the user interface 702.

The present disclosure may comprise one or more of the following features and combinations thereof.

According to one embodiment of the present disclosure, a light kit assembly for sanitizing a carrier in a drive-through banking system, comprising: a mounting plate having a back wall that extends between a first bi-fold wing and a second bi-fold wing, wherein the first bi-fold wing includes a first portion and a second portion with a first wing angle there between, and wherein the second bi-fold wing includes a first portion and a second portion with a second wing angle there between; a first UV light source assembled with one of the first portion or the second portion of the first bi-fold wing; and a second UV light source assembled with one of the first portion or the second portion of the second bi-fold wing.

In a further embodiment, one of the first or second portions of the first bi-fold wing includes a plurality of light holes; and the first UV light source includes a plurality of UVC lamps, wherein the plurality of UVC lamps is assembled with the plurality of light holes of the first or second portions of the first bi-fold wing such that the plurality of UVC lamps emit light through the plurality of light holes. In a further embodiment, the plurality of UVC lamps includes UVC LEDs.

In a further embodiment, one of the first or second portions of the second bi-fold wing includes a plurality of light holes; and the second UV light source includes a plurality of UVC lamps, wherein the plurality of UVC lamps is assembled with the plurality of light holes of the first or second portions of the second bi-fold wing such that the plurality of UVC lamps emit light through the plurality of light holes. In a further embodiment, the plurality of UVC lamps includes UVC LEDs.

In a further embodiment, the back wall and the first portion of the first bi-fold wing includes a first backwall angle there between such that the first portion opens away from the back wall, and wherein the back wall and the first portion of the second bi-fold wing includes a second backwall angle there between such that the first portion opens away from the back wall.

In a further embodiment, the back wall has a length that is longer than a length of the first bi-fold wing or a length of the second bi-fold wing.

In a further embodiment, the back wall has a length that is shorter than a length of the first bi-fold wing or a length of the second bi-fold wing.

In a further embodiment, further comprising: one or more power supply assemblies operably connected to the first and the second UV light sources.

According to yet another embodiment of the present disclosure, a method for sanitizing a carrier in a drive-through banking system having a customer station operatively coupled to a teller station wherein the carrier moves between the customer and teller stations, comprising: providing a light kit assembly having one or more UV light sources mounted on a mounting plate in one of the customer station or the teller station, the light kit assembly operatively coupled to a controller, wherein the controller is structured to control operation of the one or more UV light sources; locating the carrier in the one customer station or the teller station that includes the light kit assembly therein; closing a door of the one customer station or the teller station that contains the carrier and the light kit assembly; and operating the one or more UV light sources via the controller when the carrier and the light kit assembly are in the one customer station or the teller station such that UV wavelength light from the one or more light sources is directed towards the carrier for a designated sanitization period.

In a further embodiment, the operating the one or more light sources includes operating the light kit assembly in either an automatic sanitization mode of operation or a manual sanitization mode of operation.

In a further embodiment, the automatic sanitization mode of operation includes closing the door of the one customer station or the teller station after a period of time of inactivity of the customer unit.

In a further embodiment, the automatic sanitization mode of operation includes closing the door of the one customer station or the teller station after a designated number of uses of the customer station by one or more customers.

In a further embodiment, the operating the one or more light sources includes operating the light kit assembly in an interrupted mode of operation, wherein the interrupted mode of operation includes ceasing the one or more light sources from emitting light.

In a further embodiment, the manual sanitization mode of operation includes: activating a sanitizing lock switch operably connected to the teller unit; sending the carrier to the customer station; closing the door of the customer station that contains the carrier and the light kit assembly; and operating the one or more light sources via the controller such that UV wavelength light from the one or more light sources is directed towards the carrier for a designated sanitization period.

In a further embodiment, the designated sanitization period is about three minutes.

In a further embodiment, the light kit assembly includes an indicator light that emits light during the designated sanitization period.

According to yet another embodiment of the present disclosure, a light kit subassembly for sanitizing a carrier in a drive-through banking system that includes a customer station operatively coupled to a teller station wherein the carrier moves between the customer and teller stations, the light kit subassembly comprising: a light kit assembly including a mounting plate assembled with a first UV light source and a second UV light source, the mounting plate having a back wall that extends between a first bi-fold wing and a second bi-fold wing, wherein the first bi-fold wing includes a first portion and a second portion with a first wing angle there between, and wherein the second bi-fold wing includes a first portion and a second portion with a second wing angle there between, wherein the first UV light source is assembled with one of the first portion or the second portion of the first bi-fold wing, wherein the second UV light source is assembled with one of the first portion or the second portion of the second bi-fold wing; and a carrier pocket sized to receive the light kit assembly therein, the carrier pocket further sized and configured for installation in one of the customer or the teller stations.

In a further embodiment, one of the first or second portions of the first bi-fold wing includes a plurality of light holes, the first UV light source includes a plurality of UVC LEDs, wherein the plurality of UVC LEDs is assembled with the plurality of light holes of the first or second portions of the first bi-fold wing such that the plurality of UVC LEDs emit light through the plurality of light holes, and wherein one of the first or second portions of the second bi-fold wing includes a plurality of light holes, the second UV light source includes a plurality of UVC LEDs, wherein the plurality of UVC LEDs is assembled with the plurality of light holes of the first or second portions of the second bi-fold wing such that the plurality of UVC LEDs emit light through the plurality of light holes.

In a further embodiment, the light kit assembly includes a second mounting plate assembled with a third UV light source similar to the first UV light source, the second mounting plate assembled with a fourth UV light source similar to the second UV light source, wherein the first and the second mounting plates are positioned in a mirror-image arrangement in the carrier pocket to enable 360 degree application of UV light onto the carrier.

While the invention has been illustrated and described in detail in the drawings and foregoing description, the same is to be considered as illustrative and not restrictive in character, it being understood that only certain exemplary embodiments have been shown and described. Those skilled in the art will appreciate that many modifications are possible in the example embodiments without materially departing from this invention. Accordingly, all such modifications are intended to be included within the scope of this disclosure as defined in the following claims.

In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

It should be understood that no claim element herein is to be construed under the provisions of 35 U.S.C. § 112 (f), unless the element is expressly recited using the phrase “means for.” The schematic flow chart diagrams and method schematic diagrams described above are generally set forth as logical flow chart diagrams. As such, the depicted order and labeled steps are indicative of representative embodiments. Other steps, orderings and methods may be conceived that are equivalent in function, logic, or effect to one or more steps, or portions thereof, of the methods illustrated in the schematic diagrams. Further, reference throughout this specification to “one embodiment”, “an embodiment”, “an example embodiment”, or similar language means that a particular feature, structure, or characteristic described in connection with the embodiment is included in at least one embodiment of the present invention. Thus, appearances of the phrases “in one embodiment”, “in an embodiment”, “in an example embodiment”, and similar language throughout this specification may, but do not necessarily, all refer to the same embodiment.

Additionally, the format and symbols employed are provided to explain the logical steps of the schematic diagrams and are understood not to limit the scope of the methods illustrated by the diagrams. Although various arrow types and line types may be employed in the schematic diagrams, they are understood not to limit the scope of the corresponding methods. Indeed, some arrows or other connectors may be used to indicate only the logical flow of a method. For instance, an arrow may indicate a waiting or monitoring period of unspecified duration between enumerated steps of a depicted method. Additionally, the order in which a particular method occurs may or may not strictly adhere to the order of the corresponding steps shown. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

What is claimed is:

1. A method, comprising:
performing, with a controller of a drive up banking system, a preventative maintenance module for a control system that includes one or more of an entrance control system, a financial equipment system, a drive-thru audio system, or a pneumatic control system:
receiving, with a communication system, a current equipment identifier from equipment that includes a universal board, wherein the equipment is associated with any of the entrance control system, the financial equipment system, the drive-thru audio system, or the pneumatic control system;
determining, with the controller, whether the current equipment identifier is within a performance threshold of a historical performance identifier of the equipment;
displaying, in response to the current equipment identifier being within the performance threshold, the current equipment identifier on a user interface; and
determining, in response to the current equipment identifier not being within the performance threshold, whether the current equipment identifier is an informational event.

2. The method of claim 1, wherein in response to the current equipment identifier being within the performance threshold having been satisfied, further comprising determining a type of maintenance required for the equipment.

3. The method of claim 1, wherein the historical performance identifier includes an average life-expectancy of the equipment.

4. The method of claim 1, wherein in response to the current equipment identifier being the informational device event having been satisfied, further comprising continuing operation of the equipment.

5. The method of claim 1, wherein in response to the current equipment identifier not being the informational device event having been satisfied, determining whether the current equipment identifier is an atypical device event;
in response to the current equipment identifier being the atypical device event having been satisfied, performing a clearing workflow of a carrier between a customer unit and a teller unit of the drive up banking system; and
determining whether the clearing workflow was successful, and in response to the clearing workflow being successful changing the current equipment identifier to the informational device event.

6. The method of claim 5, wherein in response to the current equipment identifier not being the atypical device event having been satisfied, determining whether the current equipment identifier is an error device event;
in response to the current equipment identifier being the error device event having been satisfied, performing a clearing workflow of a carrier between a customer unit and a teller unit of the drive up banking system; and
determining whether the clearing workflow was successful, and in response to the clearing workflow being successful changing the current equipment identifier to the informational device event.

7. The method of claim 6, wherein in response to the current equipment identifier not being the error device event having been satisfied, determining whether the current equipment identifier is a detrimental device event; and
in response to the current equipment identifier being the detrimental device event having been satisfied, displaying the current equipment identifier on the user interface.

8. The method of claim 1, wherein the informational device event includes a current status of any of the entrance control system, the financial equipment system, the drive-thru audio system, and the pneumatic control system.

9. The method of claim 1, wherein the informational device event includes a current status of a customer unit or a teller unit associated with the drive up banking system.

10. The method of claim 7, wherein the detrimental device event corresponds to a failure in the equipment.

11. The method of claim 5, wherein the performing the clearing workflow includes:
moving, via the controller, the carrier to the teller unit;
in response to moving the carrier to the teller unit, determining whether the carrier is physically located at the teller unit; and
wherein in response to the carrier not being physically located at the teller unit being satisfied, the current equipment identifier is the atypical device event.

12. The method of claim 11, wherein the performing the clearing workflow includes:
wherein in response to the carrier being physically located at the teller unit being satisfied, then moving, via the controller, the carrier from the teller unit to the customer unit;
in response to moving the carrier to the customer unit, determining whether the carrier is physically located at the customer unit; and
wherein in response to the carrier not being physically located at the customer unit being satisfied, the current equipment identifier is the atypical device event.

13. The method of claim 12, wherein the performing the clearing workflow includes:
wherein in response to the carrier being physically located at the customer unit being satisfied, then recalling, via the controller, the carrier from the customer unit to the teller unit;
in response to recalling the carrier to the teller unit, determining whether the carrier is physically located at the teller unit; and
wherein in response to the carrier not being physically located at the teller unit being satisfied, the current equipment identifier is the atypical device event.

14. The method of claim 13, wherein the performing the clearing workflow includes:
wherein in response to recalling the carrier such that the carrier is physically located at the teller unit being satisfied, then opening, via the controller, a door of either the customer unit or the teller unit.

15. The method of claim 5, wherein the atypical device event includes determining that the carrier was not detected at the teller unit when the carrier was sent to the teller unit or the carrier was not detected at the customer unit when the carrier was sent to the customer unit.

16. The method of claim 15, further comprising:
tracking a number of detection events wherein the detection events correspond to either the carrier not being detected at the teller unit or the customer unit;
determining whether the number of tracked detection events is greater than a maximum number of tracked detection events; and
in response to the number of tracked detection events being greater than the maximum number not being satisfied, changing the current equipment identifier to the informational device event.

17. The method of claim 5, wherein the determining whether the current equipment identifier is an atypical device event includes:
moving, via the controller, the carrier to either the teller unit or the customer unit;
in response to moving the carrier, tracking a number of detection events wherein the detection events correspond to either the carrier not being detected at the teller unit or the customer unit as instructed by the moving step;
determining whether the number of tracked detection events is greater than a maximum number of tracked detection events; and
in response to the number of tracked detection events being greater than the maximum number being satisfied, changing the status of the current equipment identifier to an atypical device event.

18. The method of claim 17, wherein the maximum number is 10.

19. The method of claim 7, wherein in response to the current equipment identifier being the detrimental device event having been satisfied, determining a type of maintenance needed on the equipment.

20. The method of claim 19, further comprising:
discontinuing operation of the equipment in response to the current equipment identifier being the detrimental device event having been satisfied.
